(19) United States
(12) Patent Application Publication
(10) Pub. No.: US 2013/0065833 A1
(43) Pub. Date: Mar. 14, 2013

(54) ALKYLATED SP-B PEPTOID COMPOUNDS AND RELATED LUNG SURFACTANT COMPOSITIONS

(71) Applicants: Annelise E. Barron, Palo Alto, CA (US); Michelle Dohm, Palos Park, IL (US); Shannon L. Servoss, Fayetteville, AR (US)

(72) Inventors: Annelise E. Barron, Palo Alto, CA (US); Michelle Dohm, Palos Park, IL (US); Shannon L. Servoss, Fayetteville, AR (US)

(73) Assignee: Northwestern University, Evanston, IL (US)

(21) Appl. No.: 13/635,779

(22) PCT Filed: Mar. 21, 2011
(86) PCT No.: PCT/US11/00511

§ 371 Date: Nov. 27, 2012

Related U.S. Application Data
(60) Provisional application No. 61/315,706, filed on Mar. 19, 2010.

(51) Int. Cl. A61K 38/10; A61P 11/00; C07K 7/08

(52) U.S. Cl. 514/15.5; 530/326; 514/21.4

(57) ABSTRACT

SP-B peptoid compounds, lung surfactant compositions and related surfactant replacement therapies. Such SP-B peptoids can mimic lung surfactant protein B, and can be used in conjunction with biomimetic SP-C compounds over a range of lung surfactant compositions.

This application claims priority from application Ser. No. 61/315,706 filed Mar. 19, 2010, the entirety of which is incorporated herein by reference.

This invention was made with government support under Grant No. 2 RO1 HL067984 awarded by the National Institutes of Health, Grant No. BES-0101195 awarded by the National Science Foundation and Grant No. CHE-0404704 awarded by the National Science Foundation. The government has certain rights in the invention.

### BACKGROUND OF THE INVENTION

Lung surfactant (LS) is a functional lipid-protein mixture that coats the interior surfaces of the vertebrate lung as a film. By reducing surface tension (γ, mN m⁻¹) throughout the respiration cycle, LS minimizes the effort in breathing and stabilizes the alveolar network against collapse. LS predominantly forms an air/liquid (a/l) interfacial monolayer, but attached bi/multilayers are created as the film surface area is expanded and compressed. The composition of LS is primarily lipid by weight (˜90% including cholesterol), but the surfactant protein (SP) fraction (˜5-10%) is critical for biophysical functioning. (Hawgood, S., and Schiffer, K. (1991) Annu. Rev. Physiol. 53, 375-394. Johansson, J., Curstedt, T., and Robertson, B. (1994) Eur. Respir. J. 7, 372-391.) The two hydrophobic proteins, SP-B and SP-C (˜1-3 wt % of natural LS), are lipid-associated and sustain the efficacy of the film, promoting (i) rapid all interfacial adsorption, (ii) attainment of near-zero γ at end-expiration, and (iii) re-spreading of material at the interface throughout continuous respiratory cycles. SP-B and SP-C interactions with lipids are crucial for optimal surfactant activity, as lipid-only films have been shown to exhibit inferior in vitro and in vivo characteristics.

The absence or inactivation of lung surfactant results in atelectasis and respiratory distress syndrome. Acute lung injury (ALI) is a more complex disease resulting from a diverse set of etiologies. Lung inflammation and alterations to endogenous surfactant result in hypoxemia and decreasing pulmonary function.

Surfactant replacement therapy (SRT) is the common clinical practice of animal-derived LS administration for the treatment of infant respiratory distress syndrome (IRDS). SRTs for acute respiratory distress syndrome (ARDS) do not yet exist. Although animal-derived SRTs have been efficacious, concerns with regard to possible zoonotic infection, and difficulties in surfactant production, such as the expense of extraction and inherent batch-to-batch variability, have spurred the research and development of synthetic formulations. However, without SP-B, SP-C, or functional mimics thereof, these formulations would not match the performance of current SRTs. Peptoids, or poly-N-substituted glycines, have shown promise as mimics of SP-B and SP-C, and offer the advantages over peptides of facile synthesis, enhanced bioavailability and biostability through protease-resistance, and a longer shelf life with low propensity for irreversible aggregation in solution.

In particular, SP-B mimicry has typically involved targeting specific structural attributes of the protein believed to impart surface activity. SP-B is a net-cationic 79-residue monomer with three intramolecular disulfide bonds, and one intermolecular disulfide bond that homodimerizes SP-B in vivo. Its unresolved structure is believed to contain four to five amphipathic helices, yielding an overall helical conformation. To date, most SP-B mimics represent SP-B₁₋₂₅, the surface-active, amphipathic, and helical N-terminus of the protein. Designed sequences either exactly replicate or simplify this segment, and in the interest of mimicking SP-B's more complex structure, dimerized versions of two amphipathic helices have also been created. With its hinge-like, cationic, and amphipathic structure, SP-B can be viewed as a interfacial lipid transporter and organizer, transiently inserting into lipid layers through electrostatic and hydrophobic interactions, and facilitating folding and re-spreading of LS material at the a/l interface.

However, dimeric SP-B mimics and various other structural analogs have proven difficult and impractical to synthesize or obtain in commercial quantity or sufficient purity. As a result, there remains an on-going concern in the art to provide an alternative approach to SP-B compounds of the sort for use in pulmonary surfactant compositions, associated surfactant replacement therapies and/or, generally, for in vivo treatment of respiratory distress.

### SUMMARY OF THE INVENTION

In part, this invention can be directed to a poly-N-substituted glycine compound of a formula

A-B—X—Y—Z

wherein A can be selected from H and N-alkyl substituted glycine residues, where such an alkyl substituent can be selected from or can comprise about C₄- about C₂₄ linear, branched and cyclic alkyl moieties; B can be selected from N-alkyl substituted glycine residues, where such an alkyl substituent can be selected from or can comprise C₄- about C₂₄ linear, branded and cyclic alkyl moieties; X can be a component comprising about 3- about 9 N-substituted glycine residues, such X component as can comprise a plurality of Nₛₚₑ residues; Y can be a component comprising about 3- about 23 N-substituted glycine residues, such a Y component as can comprise residues selected from N_{Lys} and Nₛₚₑ residues, providing at least one of said residues can be N_{Lys}; and Z can be a C-terminus selected from NH₂, one and two N-substituted glycine residues selected from N_{Lys} and Nₛₚₑ.

In certain non-limiting embodiments of such a compound, each of A and B can be independently selected from N-alkyl substituted glycine residues. In certain such embodiments, each such alkyl substituent can be independently selected from linear C₈-C₂₀ alkyl moieties. Althernatively, in other embodiments, each such X residue can be an Nₛₚₑ residue. In certain such embodiments, such an X component can be 6-7 residues. Regardless of X, such a Y component can be 10-14 residues comprising a combination of N_{Lys} and Nₛₚₑ residues. In certain such embodiments, 4-6 of such residues can be N_{Lys}.

In yet other non-limiting embodiments, X and Y components can independently comprise at least one other residue selected from proline and N-substituted glycine residues, such N-substituents independently as can be selected from α-amino acid side chain moieties and carbon homologs thereof. In certain such embodiments, X and Y can independently comprise residues selected from Nₚₘ, N_{ssb}, N_{sdp}, N_{Leu}, N_{Ile}, Nₚₕₑ, N_{Trp}, N_{His} and N_{Tyr}, and combinations thereof. In certain such embodiments, Y can comprise an N_{Arg} residue. Regardless, an X component can be 6-7 residues, a Y component can be 10-14 residues and Z can be NH₂.

In part, the present invention can be directed to a poly-N-substituted glycine lung surfactant compound of a formula

A-B—X₆—Y—Z₂—Y₂—Z₄—Y—Z₂—C.

In such a compound, A can be selected from H, terminal N-substituted glycine residues and terminal N-alkyl substituted glycine residues, where such an alkyl substituent can be selected from about C₄ to about C₂₄ linear moieties; B can be selected from N-substituted glycine residues and N-alkyl substituted glycine residues, where such an alkyl substituent can be selected from about C₄ to about C₂₄ linear moieties, providing at least one of A and B is an N-alkyl substituted glycine residue; C can be a C-terminal moiety of such a compound, including but not limited to NH₂; and X, Y, and Z can also be independently selected from N-substituted glycine residues, such N-substituents as can be independently selected from α-amino acid side chain moieties and structural/functional analogs thereof and proline residues. As described elsewhere herein, such a sequence can provide such a compound a certain amphipathicity. Such structural and/or functional analogy can be considered in the context of any such α-amino acid side chain, N-substituent and/or a sequence of such N-substituted glycine residues, such structure and/or function including but not limited to charge, chirality, hydrophobicity, amphipathicity, helical structure, and facial organization. Such analogs include, without limitation, carbon homologs of such side chainsuch homologs including but not limited to plus or minus 1 or 2 methylene and/or methyl groups.

As illustrated herein, such N-substituted glycine residues can be considered in conjunction with an Nsub designation, where “sub” refers to an N-pendant substituent, such a substituent as can be an α-amino acid side chain, alkyl, or any other substituent described or referenced elsewhere herein. Such N-substituted glycine residues, substituents and corresponding designations are as would be understood by those skilled in the art made aware of this invention, for instance as shown in U.S. Pat. No. 6,887,845, the entirety of which is incorporated herein by reference.

More generally, with consideration of the preceding, whether or not helical, the peptoid compounds of this invention can comprise about 15 to about 35 residues. Such compounds can be alkylated (e.g., including one (A), two (A and B) or more N-alkyl substituted glycine residues) or non-alkylated at or about the N-terminus. Notwithstanding sequence length or alkylation, the N-terminal section of such a compound can comprise up to about 10 Nspe residues (see, e.g., FIG. 1), one or more of which can be interchanged with an Npm (pm≡phenylmethyl) residue, an Nssb (ssb≡(S)-sec-butyl) residue, an Nsdp (sdp≡(S)-1,2-dimethylpropyl) residue or a residue comprising an N-leucine (Leu), isoleucine (Ile) or phenylalanine (Phe) substituent or a substituent structurally and/or functionally equivalent thereto, with one or more other ring or aromatic-type residues optionally inserted thereon, such residues including but not limited to NTrp (Trp≡tryptophan), NHis (His≡histidine), NTyr (Tyr≡tyrosine) or Proline (Pro). Regardless, the position and number of NLys residues (see, e.g., FIG. 1) can vary, without limitation, from about 4 to about 6, one or more of which can be interchanged with an NArg (Arg≡arginine) residue.

As relates to certain such embodiments, A and/or B can be N-alkyl substituted glycine residues, with such A and/or B alkyl substituent independently selected from about C₄ to about C₂₄ linear alkyl moieties. In certain such embodiments, A can be a terminal Noct (oct=octadecyl) residue, B can be an Noct residue, C is NH₂, X can be an Nspe residue, Y can be an NLys residue, and Z can be an Nspe residue. Without limitation, such a compound can be of a formula:

Noct₂-Nspe₆-NLys-Nspe₂-NLys₂-Nspe₄-NLys-Nspe₂-NH₂.

In part, the present invention can also be directed to a pulmonary surfactant composition. Such a composition can comprise one or more of the poly-N-substituted glycine compounds of this invention, such a compound of the sort described herein or as would otherwise be understood by those skilled in the art made aware of this invention; and a lipid component selected from naturally-occurring phospholipids, non-natural phospholipids, naturally-occurring fatty acids, non-natural analogs of such fatty acids, commercially available surface-active agents and combinations thereof. Such a composition can have or provide in vitro surface activity, physiological alveolar surface activity and/or in vivo efficacy in the treatment of a mammalian subject.

In certain non-limiting embodiments, such compositions can comprise one or more other surfactant protein components including but not limited to one or more naturally-occurring surfactant proteins or biomimetic peptoid compounds (e.g., without limitation, SP-C, one or more biomimetic SP-C components and/or one or more functional analogs thereof) such compounds now or hereafter known in the art, including but not limited to peptoid compounds of the sort described in co-pending application Ser. No. 61/320,113 filed Apr. 1, 2010, the entirety of which is incorporated herein by reference.

Accordingly, this invention can be directed to a range of pharmaceutical compositions comprising one or more of the present peptoid compounds, a lipid component and/or a pharmaceutically-acceptable carrier. Such compositions can be prepared and/or formulated as would be understood by those skilled in the art made aware of this invention. One or more of the peptoid compounds of this invention, alone or together with one or more naturally-occurring and/or derived surfactant proteins, protein mimics, spreading agents or structural/functional analogs thereof can comprise about 1 wt % or less to about 20 wt % or more of such a composition, such an amount at least partially sufficient to affect and/or reduce an alveolar or in vitro air/liquid surface tension. Regardless, as illustrated below, any of the present peptoid compounds and/or related compositions can be used alone or in combination, in conjunction with one or more respiratory therapies or treatment methodologies. Without limitation, such a method can comprise providing one or more such peptoid compounds and/or related compositions; and administering such compound(s)/composition(s) using any recognized delivery technique and/or contacting a lung/alveolar interface or an in vitro air/liquid model thereof.

Accordingly, the present invention can also be directed to a method of treating a mammalian pulmonary disorder. Such method can comprise providing a mammalian subject exhibiting a physiological condition comprising a lung surfactant deficiency; and administering a composition of this invention to such a subject. Such a composition can be formulated to provide a therapeutically effective amount thereof, as would be understood by those skilled in the art made aware of this invention. In certain embodiments, together with a biomimetic SP-B compound, such a composition can comprise a component selected from naturally-derived SP-C components, biomimetic SP-C components and combinations thereof. In certain such non-limiting embodiments, such an SP-B compound can be selected from N_{oct}-Nₛₚₑ₇-N_{Lys}-Nₛₚₑ₂-N_{Lys2}-Nₛₚₑ₄-Nₛₚₑ₄-N_{Lyr}-Nₛₚₑ₂ and N_{oct2}-Nₛₚₑ₆-N_{Lys}-Nₛₚₑ₂-N_{Lys2}-Nₛₚₒ-Nₛₚₑ₄-N_{Lys}-Nₛₚₑ₂. Regardless, such a composition can be formulated as a liquid bolus, an aerosol spray or otherwise as understood in the art. Regardless, such a composition can be administered tracheally to such a mammalian subject.

### BRIEF DESCRIPTION OF THE DRAWINGS

FIG. 1. In accordance with this invention, chemical structure and side chain structures of non-limiting peptoid 3, a dialkylated variant of peptoid 1. For other representative peptoid sequences and molecular weights, see Table 1.

FIGS. 2A-B. In accordance with this invention, (A) various non-limiting side chain moieties, as can be incorporated into N-substituted glycine residues and corresponding peptoid compounds and related compositions; and (B) a schematic illustration of the sub-monomer synthetic protocol for polypeptoids. Steps 2 and 3 are simply repeated for the addition of each monomer unit. Once the full polypeptoid has been synthesized, it is cleaved off the resin with trifluoroacetic acid and purified by reversed-phase HPLC.

FIGS. 3A-B. CD and UV/Vis spectra of peptoids in methanol at room temperature (see, Table 1). (A) CD spectra for peptoids in methanol at 60 μM. λ is Wavelength (nm) and θ is Per Residue Molar Ellipticity (deg cm² dmol⁻¹). 1 (black, solid), 2 (red, dashed), 3 (blue, solid). (B) UV/Vis spectra for peptoids in methanol at 5, 50, and 100 μM. Abs is Absorbance in arbitrary units. 1 (5 mM, black, closed circles; 50 μM, black, open circles; 100 μM, black, open circles in squares), 2 (5 μM, red, closed squares; 50 red, open squares; 100 μM, red, open circles in squares), 3 (5 blue, closed triangles; 50 μM, blue, open triangles; 100 μM, blue, open squares).

FIG. 4A-B. PBS static—(A) and dynamic-mode (B) data for lipid-peptoid aqueous buffer suspensions at 37° C. Tanaka lipids alone (TL, black, circles), TL+1 (red, squares), TL+2 (blue, triangles), TL+3 (orange, diamonds), and TL+SP-B (purple, vertical crosses) in aqueous buffer (150 mM NaCl, 10 mM HEPES, 5 mM CaCl₂, pH 6.9) at 37° C. (See Tables 2 and 3 for static- and dynamic-mode data, respectively.) Data presented are representative traces, where in dynamic-mode (B), traces are after 5 minutes of cycling at the approximate adult respiratory rate of 20 cpm, and bubble expansion is clockwise from left to right.

FIG. 5. LWSB surface pressure (π) molecular Area (A) compression isotherms and FM images at 37° C. π-A isotherms are representative first compressions of a spread film on aqueous buffer (150 mM NaCl, 10 mM HEPES, 5 mM CaCl₂, pH 6.9) with a unidirectional barrier speed of 30 mm min⁻¹. Tanaka lipids alone DPPC:POPG:PA (68:22:9, wt) (TL, black), TL+1 (red, circles), TL+2 (blue, squares), TL+3 (orange, triangles), and TL+SP-B (purple, crosses). See Table 4 for isotherm 2D phase transition markers. FM images for TL and TL+1, 2, 3, SP-B₁₋₂₅, or SP-B are presented on the right at ˜40 and 55 mN m⁻¹ at 37° C. To record images, films were spiked with 0.50 mol % TR-DHPE and compressed at a barrier speed of 5 mm min⁻¹. Peptoids are present at 2.16 mol % in the lipid film, which corresponds to 10 wt % SP-B₁₋₂₅ relative to the total lipid content. Porcine SP-B is present at 0.72 mol % based on the monomer, which corresponds to 10 wt % relative to the total lipid content.

FIG. 6. LWSB surface pressure (π)-molecular Area (A) expansion isotherms at 25° C. (A) and 37° C. (B). π-A isotherms are representative first film expansions after compression to 70 mN m⁻¹ on aqueous buffer (150 mM NaCl, 10 mM HEPES, 5 mM CaCl₂, pH 6.9) with a unidirectional barrier speed of 30 mm min⁻¹. Tanaka lipids alone DPPC:POPG:PA (68:22:9, wt) (TL, black), TL+1 (red, circles), TL+2 (blue, squares), TL+3 (orange, triangles), and TL+SP-B (purple, crosses).

FIG. 7. Schematic representation of a hypothesized mode of lipid-peptoid interaction. Black circles represent DPPC, gray circles represent POPG, and white circles represent PA in the TL lipid mixture DPPC:POPG:PA 68:22:9 [wt]. The ‘insertion region’ of the peptoid helix inserts into the interfacial lipid monolayer, with the Noct hydrocarbon chains associating with the lipid acyl chains, and the amphipathic helix associating with the anionic lipid headgroups, sublayer lipid structures, and the aqueous buffer liquid subphase.

FIG. 8. Chemical structures of peptoid-based mimics of SP-B and SP-C. The eight N-terminal residues of pC contain side chains that are analogous to SP-05-12, and the remaining 14 aromatic hydrophobic residues form a helix that mimics the membrane spanning, hydrophobic helix of native SP-C. The N-terminal octadecyl amine moiety of the SP-C peptoid is a motif intended to mimic the post-translational modification of palmitoylated residues 5 and 6 in human SP-C. The SP-B mimic was designed to emulate the insertion region and helical amphipathic patterning of SP-B1-25, with the added feature of an N-terminal octadecylamine substituent.

FIG. 9A-B. Physiological indicators of pulmonary gas exchange function over time. (A) PaO2/FIO2 and (B) Blood pH over the time course of the experiment. Error bars indicate the standard error of the mean (SEM). Statistical significance indicators: * indicates p<0.05 between BLES treatment group and Tanaka Lipids (TL); + indicates p<0.05 between pC treatment group and Tanaka Lipids.

FIGS. 10A-B. Vital signs of all animals throughout the timecourse of the experiment. (A) Heart rate and (B) blood pressure at baseline measurement (BL), after lavage and before exogenous surfactant treatment (Pre-Rx), and at time points throughout the ventilation period. Error bars indicate the standard error of the mean (SEM).

FIG. 11A-C. Physiological indicators of pulmonary function. (A) Shunt fraction (B) A-a gradient, and (C) Peak inspiratory pressure (PIP) over the time course of the experiments. Error bars indicate the standard error of the mean (SEM). Statistical significance indicators: + indicates pC different from Tanaka lipids; * indicates BLES different from Tanaka lipids; # indicates pC different from pB.

FIGS. 12A-B. Surfactant pool characterization in broncheoalveolar lavage (BAL). (A) Average amounts of total surfactant, large aggregates, and small aggregates in BAL. (B) Average total protein content in the BAL of each treatment group. Error bars indicate the standard error of the mean. Statistical significance indicators: * indicates p<0.05 for the difference between the designated group and TL alone group.

### DETAILED DESCRIPTION OF CERTAIN EMBODIMENTS

Regarding the design of several non-limiting embodiments, the helicity, hydrophobicity, and cationic facial amphipathicity of SP-B₁₋₂₅ (the surface-active N-terminus fragment of SP-B) were considered, together with a minimal number of different side chains—to emulate KL₄, a simplified peptide that generically adopts the overall charge patterning of SP-B. (Cochrane, C. G., and Revak, S. D. (1991) Science 254, 566-568.) Utilizing a single-helix, peptoid-based approach, bulky, chiral, and aromatic side chains (e.g., Nspe) were chosen for increased surfactant activity relative to sequences containing chiral, aliphatic side chains. A hydrophobic helical insertion region at the N-terminus, to mimic the flexible SP-B₁₋₉ fragment, was optionally included for γ-reducing ability. A broader, more charge patterning in the amphipathic segment was also incorporated into the sequences; for instance, using Lys-like (e.g., NLys) instead of Arg-like (NArg) residues. Such considerations resulted the SP-B peptoid compounds of this invention. Representative mono-(2) and dialkylated (3) peptoids (e.g., with one or two N-(octadecyl)glycine Noct residues) were prepared and compared favorably with the prior art, as shown below.

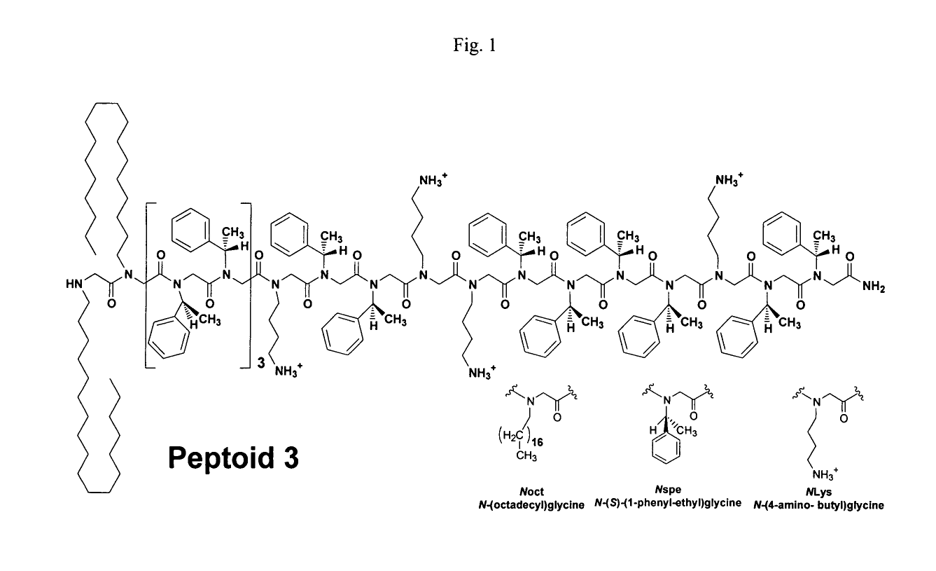

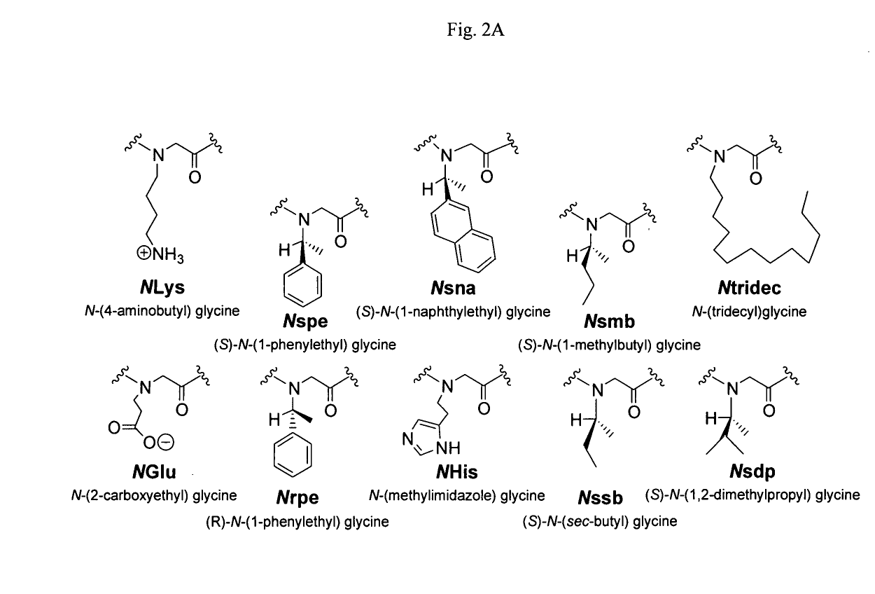

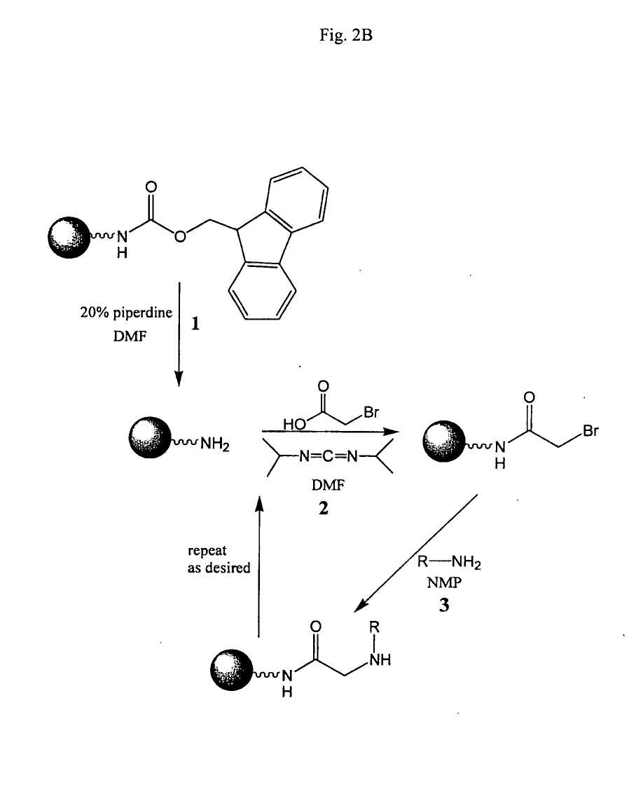

TABLE 1
Peptoid sequence information and quantitative content in the lipid
film. Side chains and structure are outlined in FIGS. 1-2A.

| | | MW (Da) | Wt % in |
|---|---|---|---|
| ID | Sequence (amino to carboxy) | Calc:Found | Film^{‡} |
| 1 | Nspe₈-NLys*-Nspe₂-NLys₂-Nspe₄-NLys-Nspe₂ | 3108.93:3109.28 | 10.8 |
| 2 | Noct-Nspe₇-NLys-Nspe₂-NLys₂-Nspe₄-NLys-Nspe₂ | 3257.26:3256.77 | 11.3 |
| 3 | Noct₂-Nspe₆-NLys-Nspe₂-NLys₂-Nspe₄-NLys-Nspe₂ | 3405.59:3405.78 | 11.9 |
| SP-B₁₋₂₅ | FPIPLPYAWLARALIKRIQAMIPKG | 2865.55:2865.30 | 9.1 |

*Underlined side chains are cationic.
Peptoids and SP-B₁₋₂₅ were purified on an RP-HPLC Long C4 Peeke column with a linear gradient of 40-80% of solvent B in solvent A over 40 minutes (solvent A is 18.2 mΩ · cm Milli-Q water with 0.1% TFA [v/v]; solvent B is acetonitrile with 0.1% TFA for 1 and SP-B_{1-25 Cys8, 11→Ala}; for 2 and 3, solvent B is acetonitrile/2-propanol 2/1 with 0.1% TFA [v/v]).
^{‡}Absolute wt % used for PBS and LWSB/FM studies

As demonstrated below, the poly-N-substituted glycine compounds of this invention, surfactant compositions and/or related methods of treatment can improve upon existing therapies and associated deficiencies. Without limitation, as compared to the prior art, various such compounds of this invention can have greater surface-adsorptive properties in a lipid film, can have greater surface-tension-reducing properties in a lipid film, and/or can be varied by residue sequence and/or N-substituent—to provide improved hydrophobicity and/or amphipathicity and/or lipid affinity.

In support thereof, representative peptoid compounds were prepared as described in Example 1 and illustrated in FIG. 2B. Variations on such synthetic techniques, N-substituents, resulting glycine residues and corresponding peptoids are as described in co-pending applications 61/320,113 (filed Apr. 1, 2010), Ser. No. 12/378,034 (filed Feb. 9, 2009) and U.S. Pat. No. 6,887,845—each of which is incorporated herein by reference in its entirety. The compounds were then structurally characterized by circular dichroism spectroscopy (CD) and ultraviolet/visible (UV/Vis) spectroscopy. The behavior of the peptoid mimics in bi/multilayer mixed lipid systems was assessed, with static and dynamic functional properties of mimics determined via pulsating bubble surfactometry (PBS). Further insight into interfacial lipid-peptoid monolayer behavior at the microscale was afforded by Langmuir-Wilhelmy surface balance studies with FM imaging (LWSB/FM). The activities of such peptoids were compared to those of porcine-derived SP-B and the N-terminal fragment SP-B₁₋₂₅.

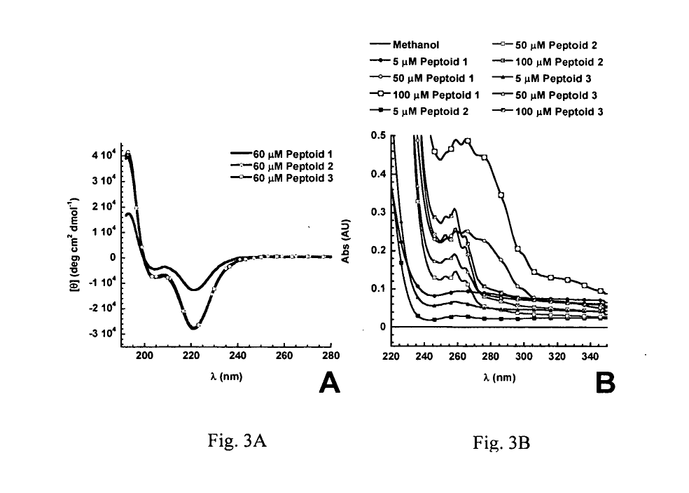

Circular dichroism spectroscopy. CD spectra were recorded for all peptoids at ˜60 μM in methanol, a lipid-mimetic environment, at room temperature (FIG. 3A). All peptoids yielded spectra characteristic of aromatic peptoid helices, with a global maximum at a wavelength (λ) of ˜193 nm, a minimum at ˜205 nm, and a global minimum at ˜221 nm. The spectra of 2 and 3 directly overlaid one another (FIG. 3A). A noticeable increase in extent of helicity was observed upon alkylation in 2 and 3 relative to 1, particularly at the global maximum and global minimum X (FIG. 3A).

Ultraviolet/Visible Spectroscopy

To further probe the spectroscopic and associative properties of peptoids in a lipid-mimetic environment, UV/Vis is spectra were collected in methanol at room temperature, at concentrations of ˜5, 50 and 100 μM (FIG. 3B). At all three concentrations, the spectra for 2 and 3 exhibited similar features, which were distinctly different from the spectrum of 1. At a λ of ˜260 nm, which surveys the environment of aromatic functional groups, the peak shape was significantly broader for 1 than 2 and 3, and there was an increased absorbance in the light scattering range of λ 300-350 nm for 1, which was lacking in 2 and 3. Most noticeable was that at each concentration, the absorbance at λ˜260 nm for the peptoids decreased significantly in the order of 1>3>2 (FIG. 3B). The changes in spectral signatures and the considerable drop in UV/Vis is absorption upon alkylation are indicative of hydrophobic interactions between the alkylated hydrocarbon chains and consequently, hydrophobic and aromatic interactions between the amphipathic, Nspe-containing helices.

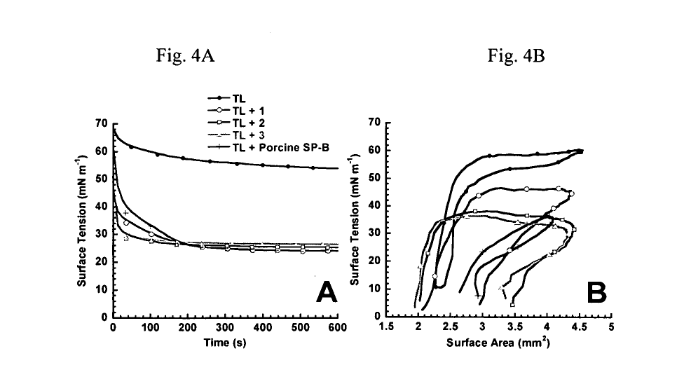

Pulsating bubble surfactometry. The adsorptive properties (γ vs. time) of lipid-peptoid suspensions at the interface of a 0.40 mm radius bubble were monitored using the PBS at 37° C. in static-mode (FIG. 4A, representative traces), and mean γ±σ at selected time intervals are presented in Table 2. Infasurf®, a clinically administered SRT derived from natural LS, typically reaches a low equilibrium γ (γ_{eq}) of ˜23 mN m⁻¹ at the all interface within 1-2 minutes on the PBS. (Seurynck, S. L., Brown, N. J., Wu, C. W., Germino, K. W., Kohimeir, E. K., Ingenito, E. P., Glucksberg, M. R., Barron, A. E., and Johnson, M. (2005) J. Appl. Physiol. 99, 624-633.) Peptoid activity in a Tanaka lipid (TL) film (e.g., 1,2-dipalmitoyl-sn-glycero-3-phosphocholine (DPPC): 1-palmitoyl-2-oleoyl-sn-glycero-3-phospho-rac-(1-glycerol) (POPG):palmitic acid (PA) 68:22:9 [wt]) was compared to the activities of porcine-derived SP-B and SP-B₁₋₂₅ in the same lipid mixture. (See, e.g., Tanaka, Y., Takei, T., Aiba, T., Masuda, K., Kiuchi, A., and Fujiwara, T. (1986) J. Lipid Res. 27, 475-485.)

The TL+SP-B suspension adsorbed to γ˜25 mN m⁻¹ in less than 5 minutes, and attained a γ_{eq} of 24 mN m⁻¹ (FIG. 4A, Table 2). The seeming inability of TL+SP-B to reach γ_{eq 24} mN m⁻¹ within one minute is likely related to the lipid composition, which only partly mimics the total lipid composition of natural LS. In stark contrast, TL adsorbed very slowly, requiring 20 minutes to reach a high γ_{eq} of ˜53 mN m⁻¹. The TL+SP-B₁₋₂₅ film (trace not shown) exhibited less rapid adsorption with an γ_{eq} of 36 mN m⁻¹ (Table 2). All lipid-peptoid mixtures dramatically enhanced the adsorption characteristics relative to TL. As suggested by the increased a of TL+1 relative to TL+2 and TL+3 up until 10 minutes (Table 2), this film exhibited slightly variable, slow adsorption rates, which were always slower than those of TL+2 and TL+3. Mono- and dialkylation greatly improved the adsorption rate of the lipid-peptoid films, with both TL+2 and TL+3 reaching ˜27 mN m⁻¹ before 2.5 minutes (FIG. 4A, Table 2). However, this increase seemed to manifest at the slight expense of low γ_{eq}, as alkylated mimic-containing films adsorbed to ˜25-26 mN m⁻¹, while TL+1 attained a γ_{eq} of ˜23 mN m⁻¹.

TABLE 2
Summary of PBS adsorption data at 37° C. in static-mode at selected time
intervals. Dried lipid-peptoid films(lipid ~1 mg mL⁻¹) were resuspended
in aqueous buffer (150 mM NaCl, 10 mM HEPES, 5 mM CaCl₂, pH 6.9)
and allowed to adsorb to the bubble interface over time. The standard
deviation of the mean (σ) is also reported.

| | γ* | | γ | | γ | | γ | | γ_{eq} | |
|---|---|---|---|---|---|---|---|---|---|---|
| | 1 min | | 2.5 min | | 5 min | | 10 min | | 20 min | |
| Film | Avg | σ | Avg | σ | Avg | σ | Avg | σ | Avg | σ |
| TL^{‡} | 61.3 | 1.9 | 59.0 | 1.8 | 56.8 | 1.8 | 54.6 | 1.8 | 52.6 | 1.7 |
| TL + | 33.9 | 5.8 | 29.4 | 4.6 | 26.5 | 3.9 | 23.9 | 1.2 | 23.2 | 0.9 |
| 1^{¥} | | | | | | | | | | |
| TL + 2 | 28.6 | 1.1 | 27.1 | 0.5 | 26.3 | 0.6 | 25.5 | 0.7 | 25.3 | 0.9 |
| TL + 3 | 27.8 | 0.9 | 27.0 | 0.8 | 26.5 | 0.8 | 25.8 | 0.9 | 25.6 | 1.4 |
| TL + | 33.5 | 2.4 | 28.3 | 1.9 | 25.7 | 0.8 | 24.6 | 0.9 | 24.4 | 1.1 |
| SP-B^{§} | | | | | | | | | | |
| TL + | 40.5 | 1.2 | 39.4 | 1.9 | 37.9 | 1.2 | 36.8 | 1.0 | 35.6 | 1.3 |
| SP-B₁₋₂₅ | | | | | | | | | | |

*Mean surface tension in mN m⁻¹.
^{‡}Tanaka lipid mixture, DPPC:POPG:PA 68:22:9 (wt).
^{¥}Mimics added at 2.16 mol %, equivalent to 10 wt % SP-B₁₋₂₅ peptide relative to the total lipid content.
^{§}Porcine SP-B added at 0.72 mol % based on monomer composition, roughly equivalent to 10 wt % relative to the total lipid content.

The maintenance of good surfactant activity during the dynamic changes in volume or film surface area at the a/l interface was assessed via bubble pulsation in PBS dynamic-mode. This method permits a simplified evaluation of in vitro dynamic film behavior. (Seurynck, S. L., Brown, N. J., Wu, C. W., Germino, K. W., Kohlmeir, E. K., Ingenito, E. P., Glucksberg, M. R., Barron, A. E., and Johnson, M. (2005) J. Appl. Physiol. 99, 624-633.) The γ-surface area (SA) data loops in FIG. 4B for each lipid-peptoid mixture are one representative pulsation cycle at 20 cycles per minute (cpm) after five minutes at 37° C., with bubble expansion in a clockwise loop direction. The mean γ_{max/min}±σ at selected time intervals are presented in Table 3.

The absence of low-γ data in some loops results from the inability of the image analysis system to trace the bubble shape in this regime. The highly compressed state of the film, which enables it to reach low γ, often causes significant bubble shape deformation, eliminating the possibility of tracing the bubble to obtain SA or employing the ellipsoidal Laplace equation to calculate γ. However, lipid-peptoid films that did not reach <1 mN m⁻¹ could be accurately traced and never exhibited significant bubble deformation. Visual, real-time bubble inspection confirmed that significant bubble deformation occurred and that γ reached near-zero in these films. Although bubble size varied slightly for every experiment, small differences in x-axis positioning (SA) had a negligible effect on γ.

TABLE 3
PBS dynamic-mode cycling data at 37° C. at selected time intervals. Dried
lipid-peptoid films (lipid ~1 mg mL⁻¹) were resuspended in aqueous buffer (150 mM
NaCl, 10 mM HEPES, 5 mM CaCl₂, pH 6.9), wherein a bubble was pulsed at 20 cpm.
The standard deviation of the mean (σ) is reported.

| | | | | | | | | | | | | | % | |
|---|---|---|---|---|---|---|---|---|---|---|---|---|---|---|
| | 1 min | | | | 5 min | | | | 10 min | | | | comp | |
| Film | γ*ₘₐₓ | σ | γₘᵢₙ | σ | γₘₐₓ | σ | γₘᵢₙ | σ | γₘₐₓ | σ | γₘᵢₙ | σ | ∇ | σ |
| TL^{‡} | 64.3 | 2.8 | 12.3 | 2.4 | 63.3 | 2.4 | 12.3 | 1.8 | 62.3 | 2.4 | 11.6 | 3.1 | 48.7 | 2.9 |
| TL + 1^{¥} | 44.9 | 1.2 | <1 | — | 46.1 | 1.0 | <1 | — | 46.1 | 1.9 | <1 | — | 26.4 | 3.6 |
| TL + 2 | 38.6 | 2.4 | <1 | — | 39.7 | 2.8 | <1 | — | 39.8 | 2.6 | <1 | — | 16.6 | 3.5 |
| TL + 3 | 34.9 | 1.6 | <1 | — | 35.2 | 2.0 | <1 | — | 35.0 | 2.2 | <1 | — | 12.2 | 3.8 |
| TL + | 36.5 | 1.1 | <1 | — | 35.8 | 1.3 | <1 | — | 36.0 | 1.9 | <1 | — | 21.0 | 2.6 |
| SP-B^{§} | | | | | | | | | | | | | | |
| TL + | 49.6 | 0.5 | <1 | — | 49.9 | 0.4 | <1 | — | 49.8 | 0.7 | <1 | — | 33.2 | 2.7 |
| SP-B₁₋₂₅ | | | | | | | | | | | | | | |

*Mean surface tension in mN m⁻¹.
^{‡}Tanaka lipid mixture, DPPC:POPG:PA 68:22:9 (wt).
^{¥}Mimics added at 2.16 mol %, equivalent to 10 wt % SP-B₁₋₂₅ peptide relative to the total lipid content.
^{§}Porcine SP-B added at 0.72 mol % based on monomer composition, roughly equivalent to 10 wt % relative to the total lipid content. No σ values are available for “<1” table entries.
∇Percent compression is defined here as 100 * [(SAₘₐₓ − SA₂₀)/(SAₘₐₓ)], where SAₘₐₓ was the maximum surface area (SA) at expansion, and SA₂₀ was the SA at which γ first reaches 20 mN m⁻¹ upon compression.

The PBS cycling loop for Infasurf® has a maximum γ (γₘₐₓ) of ˜35 mN and a minimum γ (γₘᵢₙ) near zero. This near-zero γₘᵢₙ should appear upon cycling commencement and remain indefinitely with minimal SA compression to reach near-zero γ. Similarly, the TL+SP-B film exhibited a γₘₐₓ˜36 mN m⁻¹ and near-zero γₘᵢₙ, with 21% SA compression to reach 20 mN (FIG. 4B, Table 3). The TL film demonstrated very poor surfactant activity characteristics, with a high γₘₐₓ˜63 mN m⁻¹, a γₘᵢₙ˜12 mN m⁻¹, and ˜49% compression. The addition of unalkylated 1 to the lipid film reduced the γₘₐₓ to 46 mN m⁻¹ and the γₘᵢₙ to near-zero immediately upon pulsation. The percent SA compression was also reduced to ˜26%. The γₘₐₓ and percent SA compression further decreased upon mono- (2), and then dialkylation (3), of the peptoid helix, with 3 reaching a γₘₐₓ slightly lower than that of TL+SP-B, but matching that of Infasurf® (˜35 mN m⁻¹), and attaining a surprisingly low 12% SA compression to reach 20 mN m⁻¹ (Table 3). Also of note are the differences in bubble shape between TL+SP-B and the TL+peptoid films, where the loop hysteresis was much larger for peptoid-containing films (FIG. 4B). The significance of hysteresis has not yet been established, but in lipid-peptoid films, a large degree of hysteresis generally results in less compression to reach near-zero γ.

In peptoids, the degree of hysteresis in the PBS loop could be associated with the amount of material that is retained close to the monolayer, where the reincorporation of these attached components are important for the re-expansion process as film surface area is increased upon inhalation. Structurally, this attached material may be in the form of bilayers.

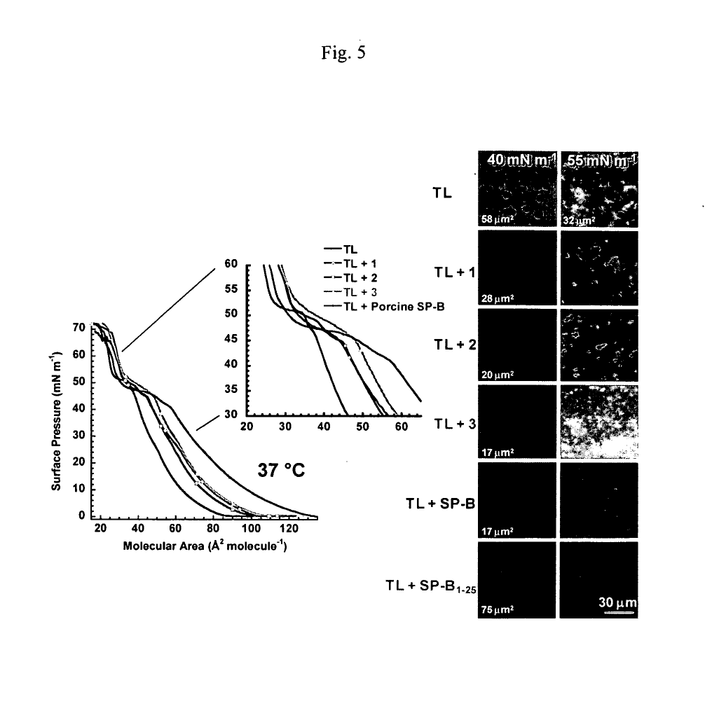

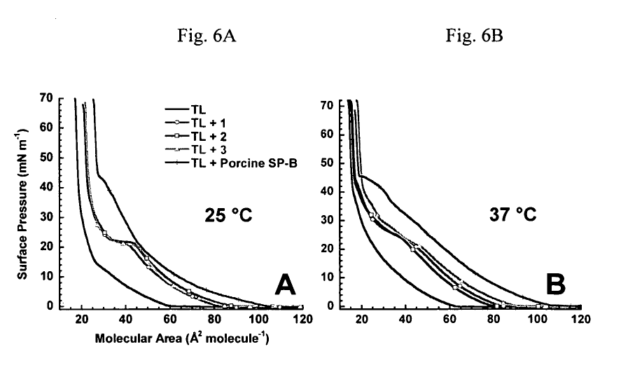

Langmuir-Wilhelmy surface balance studies and epifluorescent microscopic imaging. Surface pressure (π, mN m⁻¹) molecular area (A, Å² molec⁻¹) representative first compression and expansion isotherms (FIGS. 5 and 6, respectively) and representative FM images (FIG. 5) for TL+peptoid films spread at the all interface were obtained on a custom-built LWSB and performed at 25 and 37° C. For 25° C. compression isotherms and FM images, see FIG. 5SM. The isotherm features of the TL+peptoid films herein were compared to TL+SP-B and TL+SP-B₁₋₂₅ films (FIG. 5, Table 4). For mean liftoff data and 2D first compression isotherm features±σ, at both 25 and 37° C., see Table 4.

For the TL+SP-B film at 37° C. (FIG. 5, Table 4), the isotherm exhibited an early (high) ‘liftoff’ area, or the A at which π first measurably increases from zero. After a procession from the liquid-expanded (LE) to liquid-condensed (LC) phases, the film reached a high collapse π of ≧70 mN m⁻¹, or near-zero γ (FIG. 5). The magnitude of the increase in liftoff A with an additive, relative to the lipid-only film, correlates with the molecule's ability to crudely organize an adsorbed interfacial structure. This effect is recognized by a measurable increase in π, and is considerably affected by the additive size, which alters the available area per molecule (A). In addition, a pronounced, extended plateau occurred at ˜40-50 mN m⁻¹ in TL+SP-B films (FIG. 5). The changes in monolayer/multilayer structure in the plateau region are still well-debated, but the plateau's presence is a defining characteristic of SP-B- (and SP-C)-containing films, and corresponds to reversible exclusion (“squeeze-out”) of material from the monolayer into attached structures below or above the surface. The isotherm for TL+SP-B₁₋₂₅, previously published, exhibited a later liftoff area than TL+SP-B with a less pronounced plateau at both temperatures (Table 4), a result likely due to the smaller size of the molecule as well as a decrease in behavior relative to the full-length protein.

At 37° C., all films exhibited a high collapse of it 70 mN m⁻¹. The TL film exhibited a later liftoff and less pronounced plateau than any of the lipid-peptoid films (Table 4), while TL+SP-B demonstrated the earliest liftoff and most pronounced plateau.

TABLE 4
LWSB first compression isotherm 2D phase transition markers. Mean π-A
isotherm data from first compressions of a spread film on aqueous
buffer (150 mM NaCl, 10 mM HEPES, 5 mM CaCl₂, pH 6.9)
with a unidirectional barrier speed of 30 mm min⁻¹.
Standard deviation of the mean (σ) is reported. Liftoff is defined
as the molecular area (A) at which the surface pressure (π) first
measurably increases from zero to 1 mN m^{-1.}

| | 25.0 ± 1.5° C. | | | | | 37.0 ± 1.9° C. | | | | |
|---|---|---|---|---|---|---|---|---|---|---|
| | | | | Plateau | | | | | Plateau | |
| | Liftoff | Kink | | Length | | Liftoff | Kink | | Length | |
| Film | (A*) | (A) | (π^{‡}) | (A) | (π) | (A) | (A) | (π) | (A) | (π) |
| TL^{‡} | 84₍₄₎ | 63 | 5 | 5 | 7 | 95 | 49 | 26 | 11 | 11 |
| TL + | 107 | 79 | 6 | 7 | 8 | 113 | 60 | 27 | 14 | 11 |
| 1^{¥} | | | | | | | | | | |
| TL + | 109₍₃₎ | 81 | 5 | 9 | 9 | 110 | 62 | 26 | 14 | 10 |
| 2 | | | | | | | | | | |
| TL + | 107 | 79 | 5 | 10 | 10 | 116₍₅₎ | 63₍₅₎ | 26 | 17 | 11 |
| 3 | | | | | | | | | | |
| TL + | 120 | 94 | 5 | 11₍₃₎ | 10 | 132₍₅₎ | 69 | 26 | 24₍₃₎ | 12 |
| SP-B^{§} | | | | | | | | | | |
| TL + | 114 | 92 | 4 | 11 | 11 | 118₍₅₎ | 64 | 25 | 13 | 13 |
| SP-B₁₋₂₅ | | | | | | | | | | |
| σ^{δ} | ≦2 | ≦3 | ≦1 | ≦2 | ≦2 | 3 | ≦3 | ≦2 | ≦2 | 1 |

*Mean molecular area expressed in Å² per molecule.
^{||}Mean surface pressure expressed in mN m⁻¹.
^{‡}Tanaka lipid mixture, DPPC:POPG:PA 68:22:9 (wt).
^{¥}Mimics added at 2.16 mol %, equivalent to 10 wt % SP-B₁₋₂₅ peptide relative to the total lipid content.
^{§}Porcine SP-B added at 0.72 mol % based on monomer composition, roughly equivalent to 10 wt % relative to the total lipid content.
^{δ}For clarity, the standard deviation of the mean (σ) for each type of value is collectively reported at the base of the column, with exceptions in the table listed as “a_{(b),}” where a is the mean and b is the σ.

All TL+peptoid isotherms possessed earlier liftoff areas relative to the TL only film, which are indications of increased surface activity and the presence of an additive. The isotherm features for all three lipid-peptoid films were very similar up until the plateau region (FIG. 5), with no significant differences in liftoff area among them. However, distinct differences were apparent in the size and shape of the plateau regions, where TL+3 had the most pronounced plateau of any of the lipid-peptoid films, second only to TL+SP-B (Table 4).

To gain further insight into the 2D phase morphology of the lipid-peptoid films in the plateau region, FM images were recorded with 0.50 mol % TR-DHPE spiked into the TL or TL+peptoid solution, and are presented below and above the plateau region, i.e. at ˜40 and 55 mN m⁻¹, respectively, at 37° C. (FIGS. 5) and 30 and 60 mN m⁻¹. TR-DHPE is a bulky, fluorescently headgroup-labeled lipid that is size-excluded from the ordered phase of the monolayer upon sufficient lipid packing during film compression. Therefore, apparent dark regions of the monolayer correspond to liquid-condensed (LC) domains, while brighter regions represent the disordered liquid-expanded (LE) phase. Particularly bright spots in images signify sub- or super-monolayer protrusions of unknown composition that are excluded from, yet still associated with, the monolayer, known here as “bright protrusions.”

In these films, LC domains formed at 6-7 mN m⁻¹ at 25° C. and 28-35 mN m⁻¹ at 37° C.; bright protrusions, if present, occurred at 46-48 MN m⁻¹ at both 25° C. and 37° C. Below the plateau regime at ˜40 mN m⁻¹ and 37° C., the most perceptible lipid phase morphology differences among films were in the size of the dark LC domains surrounded by the brighter fluid LE phase (FIG. 5). The LC domains in the TL film were ˜58 μm², with larger LC domains for TL+SP-B₁₋₂₅ (˜75 μm²) and the other films ranked in the order of decreasing LC domain size 1>2>3≅SP-B. In some experimental runs, the LC domains for 2 and 3 were very difficult to image due to their small size. At ˜55 mN m⁻¹, the TL film retained LC domains of smaller size and density than at ˜40 mN m⁻¹ (˜32 μm²), with the added presence of bright protrusions, which predominantly resided in the LE phase. In contrast, in the lipid-peptoid and lipid-SP-B/SP-B₁₋₂₅ films at ˜55 mN m⁻¹, the LC domains seemed to be quite sparse or even vanish (FIG. 5). The disappearance of LC domains at physiological temperature has been previously observed in other lipid films containing aromatic peptoids.

Interestingly, the TL+SP-B film displayed clustered dark areas, which appeared to be in a more aggregated state than a typically rounded or flower-like lipid-only LC domain (FIG. 5). The other significant changes in lipid phase morphology at higher π were evident in the bright protrusion formation and patterning, which for TL+1, were rather clustered in aggregate-like structures, for TL+2, were more distinctly rounded and separated protrusions, and for TL+3, were very bright and more evenly dispersed in round protrusions (FIG. 5). In TL+SP-B₁₋₂₅, the bright protrusions were the sparsest. In TL+SP-B, the bright protrusions were more continuously clustered between dark regions and not identified as particularly bright and round-shaped as in TL+3.

To probe the lipid-peptoid interactions upon film expansion, isotherms were collected after the first compression to ˜70 mN m⁻¹, and are presented as representative expansion isotherms at 25 and 37° C. (FIG. 6). At 25° C. (FIG. 6A), the TL film exhibited a small kink at 18±5 mN m⁻¹, while the TL+SP-B film had a brief “plateau region” at 44±2 mN m⁻¹ and a small kink at 23±1 mN However, the TL+peptoid isotherms were similar to each other, with very pronounced plateaus beginning at ˜25 mN m⁻¹ and ending at ˜20 mN m⁻¹. This type of “plateau” has been suggested, in the literature, as a phase transition representing peptide reinsertion into the lipid film, and is indicative of lipid-peptoid structural reorganization at the interface. At 37° C. (FIG. 6B), the TL expansion isotherm was very similar to that at 25° C., as was that of TL+SP-B. Interestingly, the pronounced plateau in the three TL+peptoid films at 25° C. became longer and more gradual at 37° C., extending from ˜43 mN m⁻¹ to ˜20 mN m⁻¹, and more closely resembling that of the TL+SP-B film. The reinsertion of material to the interface is believed to be a factor in sustained surface activity of the LS film, and it was evident that the temperature (and hence fluidity) of the lipids significantly affected the shape of the lipid-peptoid isotherm “plateau” and, perhaps, a mechanism of subsequent incorporation of associated material back into the film.

With respect to several such embodiments, the present work departs from traditional approaches to SP-B peptidomimetics by combining N-terminus alkylation with an aromatic, SP-B-like amphipathic peptoid helix. Resulting compounds noticeably improved in vitro surface activity performance in a tri-component lipid film. As shown above and discussed more thoroughly below, such alkylated SP-B peptoid-based mimics demonstrated substantial improvement on three criteria typically considered in the context of LS activity: rapid adsorption, reduction in surface tension, and sustained surface activity through multiple pulsation cycles—thereby, indicating that material is effectively re-spread.

The increase in surface activity of 2 and 3 relative to 1 correlated with a significant increase in extent of helicity (CD) in a lipid-mimetic solvent (FIG. 2A). Note that it is not uncommon for an increase in helicity to occur upon attachment of a fatty acid chain in peptides, and is believed to result from an increase in helical stability provided by hydrophobic interactions between the hydrocarbon chain and the peptide helix. Interestingly, the UV/Vis is spectral signatures for 2 and 3 had significantly decreased absorbances at λ˜260 nm (FIG. 2B) relative to 1. Because absorbance at λ˜260 nm resulted solely from the aromatic Nspe side chains in the peptoid helix, the observed effect could be similar to that which occurs between double-stranded and denatured DNA. Without limitation, once alkylated, peptoids may engage in intermolecular chain-chain hydrophobic interactions that facilitate intermolecular aromatic residue ‘stacking,’ in turn causing peptoid self-assembly that ‘buries’ the aromatic residues and subsequently lowers UV absorbance. These potential peptoid-peptoid associations may stabilize the helical motif as well as impart surface activity to a surfactant film, where assembled alkylated peptoids collectively anchor excluded material to the interfacial monolayer via multiple alkyl chains, thereby increasing adsorptive properties and facilitating re-spreading of material.

The enhanced ability of the lipid-peptoid mixtures to increase the adsorption rate and further promote surface tension reduction upon alkylation were demonstrated using the PBS. A setback in surface activity of mimic 1 was the variable, slow adsorption rate in static-mode on the PBS, where aliphatic peptoid variants exhibited a high adsorption rate. It is known that LS adsorption is a two-step process, comprising an initial adsorption of dispersed surfactant to the interface, followed by the perturbation and reorganization of the film as new material (likely ruptured liposomes) is added. Upon an increase in aliphatic hydrophobicity via alkylation, the adsorption rates of 2 and 3 were distinctly improved, suggesting that the speed of this process was enhanced considerably. In an aqueous buffer suspension, these hydrophobic, surface-active molecules would be very attracted to the interface, and the increased associative properties of the alkylated peptoids, as evidenced by UV/Vis is, may further facilitate rapid adsorption. The SP-B-containing film's seemingly slower adsorption rate could be caused by a decrease in the rate of diffusion to the interface with increases in molecular size. Interestingly, alkylation seemed to slightly increase the γ_{eg} reached, reminiscent of aliphatic peptoids that exhibited considerably higher γ_{eq} in these lipid films.

As discussed above, maintaining a reduced γₘₐₓ throughout respiration and near-zero γₘᵢₙ upon film compression is a consideration for an effective, functional LS. All lipid-peptoid films reached near-zero γₙₐ immediately upon bubble pulsation, and retained this value for the time tested, ˜10 minutes. (The importance of near-zero γₘᵢₙ at minimum SA may be over-emphasized relative to a reduced γₘₐₓ at maximum SA; for instance, two well-known peptide mimics considered to be surface-active, KL₄ and SP-B₁₋₂₅₅ exhibited γₘₐₓ in the 48-50 mN m⁻¹ range in a TL film.) The γₘₐₓ of the aromatic peptoid helix-containing film was vastly improved upon alkylation, with additional incremental improvements for mono-, and then dialkylation. The γₘₐₓ reached for 2 and 3 matched that of the TL+SP-B film, thus surpassing the surface activity of all single helix peptide and peptoid SP-B mimics developed to date. The percent compression to reach 20 mN m⁻¹ was also significantly reduced upon alkylation. (However, the hysteresis and loop shape of the lipid-peptoid films appeared somewhat different from those of TL+SP-B.)

Information about the ability of peptoids to insert into interfacial films, to affect segregation pattern morphology, and to increase tension-active properties were obtained through quasi-equilibrium compression and expansion of spread films on the LWSB and “in situ” FM. The increased liftoff area of the SP-B-containing film relative to those of the lipid-peptoid films is attributed to the sheer size of the molecule, which is present in the mixture in both monomer and dimer form. The N-terminus alkylation in 2 and 3 did not significantly alter liftoff when compared to 1. Given that the base structural attributes of the helix remained largely unchanged upon alkylation, this result is strong evidence that the alkyl chains of the peptoids were well-incorporated into the lipid acyl chains of the film, translating to nearly the same area occupied per molecule at the interface.

Indications of superior surface activity upon alkylation are also present in the plateau regime (high π, low γ) of the compressed film, where alterations in the shape, size, and phase segregation morphology of the film are most evident. The very pronounced plateau of the SP-B-containing film demonstrates extensive structural reorganization and 3D folding of material, in and below the interfacial monolayer, which allows the film to reach high TC without substantial loss of material to the subphase. Therefore, a change in shape or increase in plateau size, as demonstrated with 2 and 3, pinpoints to an increase in behavior relative to 1. In addition, the decrease in LC domain size and increase in the density of small bright protrusions in FM images further suggests improvements in film organization upon alkylation. Small LC domains are believed to minimize line tension at the phase boundaries and increase the feasibility of film refolding and re-spreading during increases or decreases in TC, respectively.

The idea that temperature affects peptoid inclusion into the lipid film may be obvious given the increases in liftoff area and plateau size at higher temperature. However, upon film expansion, a marked phase transition or ‘plateau’ is present in lipid-peptoid films at lower temperature, which does not occur when SP-B is included. This transition may correspond to a different mechanism of reincorporating excluded peptoid or lipid-peptoid material as the available trough area increases. Surprisingly, at 37° C., near the T_{c} of the lipid film, the lipid-peptoid expansion isotherm ‘plateaus’ resembled more closely that of the TL+SP-B film. Because efficient reorganization and reincorporation of excluded material upon expansion directly relates to re-spreadability, they are considered critical features of LS films. The considerable ‘plateau’ changes in lipid-peptoid films with increasing temperature suggest that SP-B-containing film properties may still be superior; other structural attributes of SP-B are probably necessary in peptoids to achieve optimal film re-spreadability.

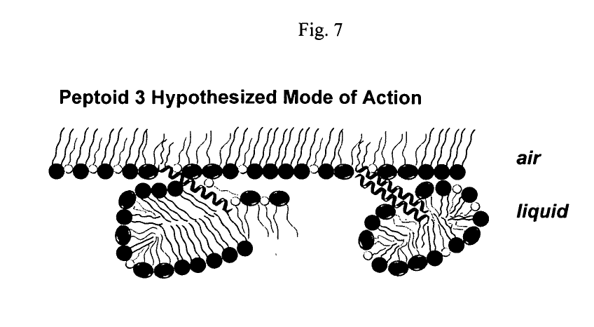

Protein lipidation, peptide-fatty acid conjugation, S-palmitoylation, and peptide amphiphiles have all been utilized in nature and by researchers to enhance biological activity. Attaching a lipid-like chain facilitates interactions with lipid membranes, allows for intra- and intermolecular associations, and functions to mediate protein trafficking and stability. In this case, the question to be answered is what the role of N-terminus alkylation is once attached to an SP-B-like amphipathic peptoid helix. Without limitation to any one theory or mode of operation, one hypothesis for a molecular mechanism of action is outlined in the graphic of FIG. 7. The alkylated chain(s) function to extend the insertion region of 1, thus ensuring that the molecule is attached to the interface, or remains predominantly lipid-associated. It is far-reaching to suggest that these simple amphipathic helices completely mimic the homodimerized natural SP-B molecule in its structure or function. The ability of these molecules to mimic SP-C is even less likely, as the facially amphipathic charge patterning and lack of high hydrophobicity in the helix prohibit any possibility of spanning a lipid bilayer or sustaining an attached surfactant reservoir. However, this alkylated amphipathic helix may be capable of remaining inserted into lipid moieties, transporting lipids to and from the interface, and in the case of 3, connecting a monolayer and a bilayer through multiple alkylated chain ‘anchors.’ These molecular actions would increase adsorptive properties, reduce the surface tension, and enhance re-spreadability of the film.

The necessity of mono- vs. dialkylation was also addressed in this study, as 3 retained a lower γₘₐₓ during cycling (which was more stable than 2, as evidenced by the decrease in σ (Table 3)), and a more pronounced plateau on the LWSB, relative to TL+2. Because a facially amphipathic helix would prefer to associate with the lipid headgroups and aqueous subphase, it is reasonable to speculate that dialkylation is necessary to fully anchor the insertion region into a lipid layer. Monoalkylation could permit conformational flexibility or intramolecular associations rather than incorporation into the lipid film. The decreased UV/Vis is absorbance at ˜60 nm of 2 relative to 3 may be an indication of this.

As demonstrated herein, through multiple in vitro surface activity tests in a tri-component lipid film, N-terminus alkylation strikingly improves the surfactant activity of a single, aromatic, and amphipathic peptoid helix mimicking SP-B—presumably, by improving the insertion ability of the peptoid into the fluid phase of the lipid film. Additional benefit in dialkylation vs. monoalkylation was also observed. These results imply that a similar strategy could be employed to augment the surface activity of single amphipathic helix peptide SP-B mimics that have not been efficacious enough for introduction to the pharmaceutical market. The synthesis of such N-terminus alkylated amphipathic helices, peptide or peptoid, is a feasible, cost-friendly, and an attractive alternative to generating mass quantities of dimerized or otherwise structurally complex SP-B-like mimics.

Accordingly, such SP-B compounds and related compositions can be employed over a range of lung surfactant replacement or associated respiratory therapies. Without limitation, such compounds and/or formulations are believed to be useful for treatment of or as a supplement to treatment of ARDS, IRDS, meconium aspiration syndrome, pneumonia, sepsis, lung injury, bronchopulmonary, dysplasia, asthma, cycstic fibrosis, idiopathic interstitial pneumonias, tuberculosis, and other bacterial and/or viral infections of the lung.

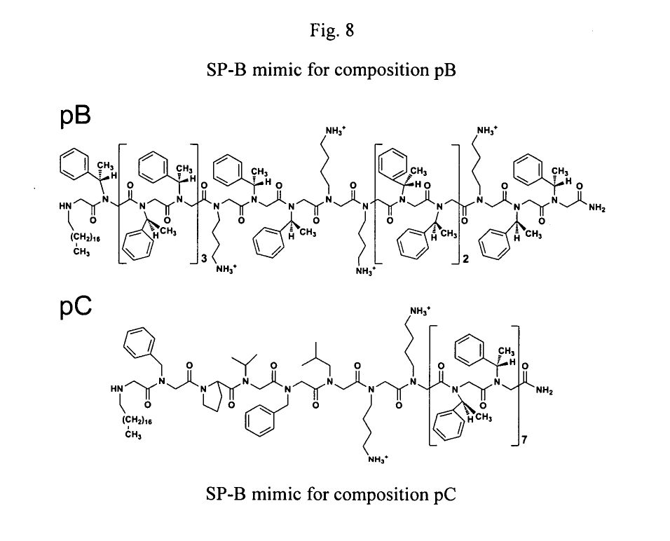

To demonstrate the use of such compounds and related compositions, a study was conducted examine the ability of peptoid-based SP-B and/or SP-C compositions (termed pB and pC, respectively, for purpose of the study) to mitigate deleterious physiological and biochemical responses associated with ALI. Compositions pB and pC were designed with representative peptoid compounds that mimic the overall hydrophobicity, amphipathicity, and helical structures of SP-B and SP-C, respectively. Compounds selected were N-terminally C₁₋₈-alkylated to mimic the palmitoyl moieties of natural SP-C, a feature known to improve in vitro surface activity. (SP-B peptoid 2, Table 1, above; and a structurally-related SP-C peptoid, FIG. 8.) The hypothesis was that such peptoid-enhanced surfactant compositions could demonstrate in vivo efficacy in an animal model of ALI that matches or exceeds that of an animal-derived surfactant.

While the extensive alveolar network and capillary vasculature of the pulmonary parenchymal tissue are critical to achieving efficient gas exchange, these delicate structures are highly susceptible to systemic pathogens and environmental toxins. A broad spectrum of direct pulmonary insults and indirect systemic maladies results in lung surfactant deficiency and dysfunction, which leads to ALI. There is currently no cure for ALI, and while exogenous surfactant treatment as part of a multimodal therapy has been shown to mitigate symptoms of the disease for a subset of patients, outcomes of clinical trials have been varied. However, the present invention provides a novel technology platform with characteristics amenable to the treatment of ALI. This inaugural study, designed to investigate the in vivo efficacy of peptoid-based surfactants, demonstrates, using the lung lavage model of ALI, that lung surfactant replacements, in accordance with this invention, can improve physiological and biochemical outcomes to an equivalent or greater extent than treatment with animal-derived surfactant. As discussed, below, peptoid-enhanced surfactant preparations demonstrated statistically significant, immediate (within 10 minutes of treatment) and/or sustained (10 minutes 2 hours) improvements in PaO₂/FIO₂, shunt fraction, a-A gradient, and PIP. This is an encouraging result for biomimetic surfactants as it marks the first reporting of peptoid-enhanced surfactant compositions demonstrating in vivo efficacy.

With reference to examples 6-15, this study evaluated the in vivo efficacy of peptoid-based SP-B and SP-C mimics formulated separately and in combination. Tanaka lipids was selected as the lipid carrier for these representative synthetic formulations based on its similarity to the lipid/fatty acid component of natural surfactant and superior in vitro surface activity compared to other lipid formulations. Though the peptoid-enhanced surfactants utilized contain a higher amount of protein mimic (˜10 wt %) relative to the quantities of SP-B and SP-C found in extracted surfactant (˜0.5-3 wt % each), this is reasonable because peptoids pB and pC (20 and 22 residues, respectively) represent only a portion of the natural proteins' structures (79 and 34 residues for SP-B and SP-C, respectively).

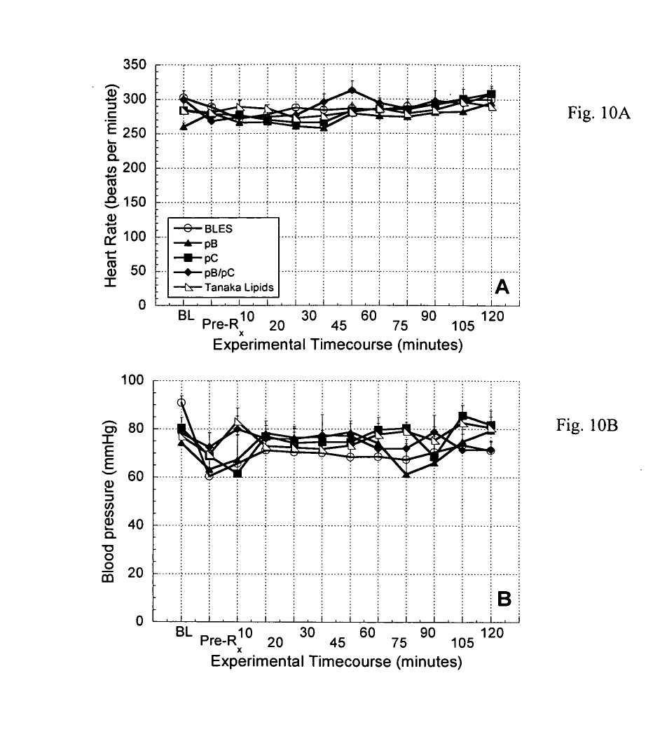

The study utilized a lung lavage model of lung surfactant deficiency in adult rats, a model that is well-characterized, accepted in the art and has previously been shown to respond to animal-derived surfactant preparations. The average heart rate and blood pressure of the various treatment groups showed no notable difference among any of the groups (FIG. 10). The average post-lavage decrease in the PaO₂ and increase in PIP showed that pulmonary gas exchange and lung compliance were significantly and uniformly damaged, a condition associated with ALI. Accordingly, as discussed more fully below, this animal model of surfactant deficiency captures most aspects of the pathophysiology associated with ALI (i.e. surfactant alterations) and was deemed well-suited for direct comparison of surfactant preparations.

The five physiological responses measured in this study consistently showed that the negative control treatment group (Tanaka lipids) resulted in the least improvement in pulmonary function. FIGS. 2 and 3 show that the Tanaka lipids alone formulation neither achieved the same initial degree of recovery, nor effectively maintained activity throughout the observation period. The notable and consistent improvement in physiological response to peptoid-enhanced Tanaka lipid formulations compared to the lipid carrier alone provide evidence for the bioactivity of peptoids to affect improved outcomes using a lung lavage model of ALI.

Whereas all peptoid-containing surfactant compositions improved physiological lung function, there were differences in the responses to the individual preparations. The pC composition demonstrated a more significant initial improvement in physiological responses and exhibited sustained benefit throughout the recovery period, compared to the other preparations. The pB composition treatment group, however, consistently had a less significant impact on measured outcomes, and on average appeared only marginally better than Tanaka lipids.

A second observation regarding the responses of the individual preparations lies in comparing the performance of compositions pB and pC formulated separately to that of the pB/pC combination composition. By all physiological measures, the pC treatment group achieved a more favorable outcome than did the pB treatment group. Interestingly, however, the pB/pC group achieved the best sustained response in PaO2/FIO2, shunt fraction, and A-a gradient. Without limitation to any theory or mode of operation, variability in the dynamic in vivo environment and lipid composition can influence the extent to which proteins and protein mimics interact. Because the synergistic interaction of protein mimics is dependent on both their chemical structures and the conditions in vivo, it is difficult to generalize observations relevant to a particular system. In this study, however, co-dosing pB and pC in the pB/pC composition enabled a better sustained response in some physiological outcomes over the two hour recovery period.

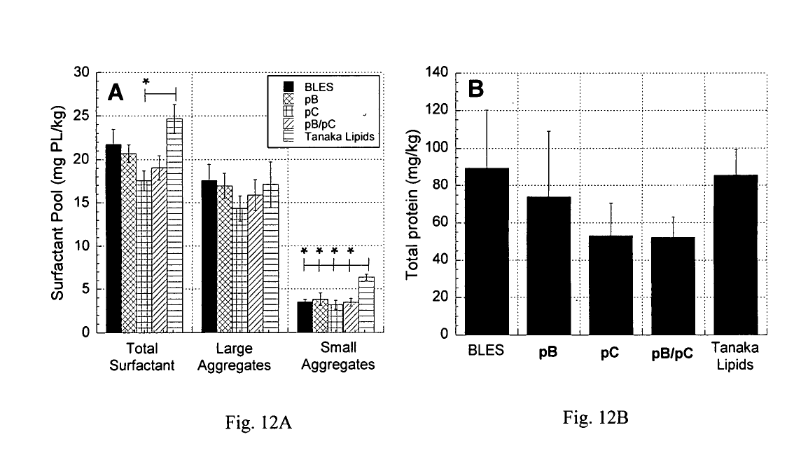

The way in which exogenously administered surfactant is metabolized is another factor that can influence its efficacy. Surfactant delivered to the airspace can subsequently be taken up by alveolar type II cells for recycling or by alveolar macrophages for degradation. In addition, within the airspace, exogenous surfactant can be converted from the active large aggregates to inactive small aggregates. These processes would all impact the efficacy of the exogenous material, and the surfactant pool characterization at the end of the ventilation period provided some insight into these effects. The data showed that the Tanaka lipids treatment group had a larger total surfactant pool than any other group and was statistically different from that of the pC treatment group (FIG. 12A). This difference could be due to disparate surfactant uptake rates for the various surfactant preparations. Because the Tanaka lipid formulation contains no proteins or protein mimics, it is possible that it may not be as readily taken up by type II cells. The rate of conversion from large to small aggregates within the surfactant pool has also been shown, in the literature, to increase under conditions pervasive in an injured lung: 1) increased protease activity, 2) altered surfactant composition, and 3) dynamic changes in surface area due to mechanical ventilation. Injured lungs, therefore, often exhibit an increased amount of total surfactant and a concomitant increase in the less surface-active Small Aggregates. FIG. 12 B shows that indeed the small aggregate component of the BAL from the Tanaka lipid treatment group was statistically greater than that of any other group. Without limitation, the increase in total surfactant of this group appears to be due to primarily an increase in the less active Small Aggregates.

The results of this in vivo study demonstrate that peptoid-enhanced lung surfactant replacements exhibit promising bioactivity and can improve physiological and biochemical outcomes using the lung lavage model of ALI. Such results are significant because biomimetic exogenous lung surfactants afford several advantages over animal-derived surfactant replacements. As mentioned above, the high cost of natural surfactant coupled with the large quantities required to treat adults for ALI can make treatment prohibitively expensive. Moreover, the use of a biomimetic surfactant avoids the risk of immune response that is inherent with animal-derived products. Biomimetic surfactants of this invention also offer the possibility of a “designer” treatment customized to mitigate specific types of surfactant dysfunction or deactivation induced by the myriad of clinical maladies that result in ALI. Accordingly, additives can be included in a synthetic formulation, not only to improve surface activity, but also to prevent surfactant inhibition, regulate surfactant homeostasis, control inflammatory response and treat bacterial and viral infections (e.g., antibiotic and/or antiviral agents). Finally, peptoids designed as biomimetics specifically exhibit secondary structure that makes them less prone to aggregation, which can result in enhanced shelf-life and facilitates synthesis and purification.

Such results, for the first time, demonstrate lung surfactant compositions of this invention, utilizing SP-B and SP-C biomimetic compounds of the sort described herein, can improve physiological and biochemical outcomes to an extent equivalent to or better than animal-derived surfactant. While all peptoid-enhanced compositions evaluated tended to improve outcomes compared to treatment with the lipid carrier alone, a pC composition exhibited the best and most sustained in vivo response.

### Examples of the Invention

The following non-limiting examples and data illustrate various aspects and features relating to the biomimetic lung surfactant compounds, compositions and/or methods of the present invention, including the preparation of various biomimetic SP-B compounds as are available through the synthetic methodologies described herein. In comparison with the prior art, the present compounds, compositions and/or methods provide results and data which are surprising, unexpected and contrary thereto. While the utility of this invention is illustrated through the use of several such compounds and N-substituted glycine residues incorporated therein, it will be understood by those skilled in the art that comparable results are obtainable with various other residue combinations, corresponding lung surfactant compounds and related compositions, as are commensurate with the scope of this invention.

Materials—Peptide and peptoid synthesis reagents and supplies were purchased from Applied Biosystems (ABI) (Foster City, Calif.) and Sigma-Aldrich (Milwaukee, Wis.). Fmoc-protected amino acids and resins were obtained from EMD Biosciences (NovaBiochem, San Diego, Calif.). Primary amines (highest purity and enantiomeric excess available, FIG. 1), di-tert-butyl dicarbonate (Boc), and palmitic acid (PA) were purchased from Aldrich. All salts, and solvents acetonitrile, 2-propanol, chloroform, methanol, and trifluoroacetic acid (TFA), HPLC grade or better, were obtained from Fisher Scientific (Pittsburgh, Pa.). DPPC and POPG were purchased from Avanti Polar Lipids (Alabaster, Ala.) and used as received. Texas Red®, 1,2-dihexadecanoyl-sn-glycero-3-phosphoethanolamine, triethylammoni-um salt (TR-DHPE) was obtained from Molecular Probes (Eugene, Oreg.). All chemicals were used without further purification. Water was Milli-Q 18.2 mΩ·cm quality.

### Example 1

### Protein Extraction and Peptide and Peptoid Synthesis and Purification

Porcine SP-B was gifted by Prof. Jesús Pérez-Gil (Madrid, Spain), isolated from minced porcine lungs as described previously. (See, Perez-Gil, J., Cruz, A., and Casals, C. (1993) Biochim. Biophys. Acta 1168, 261-270.) The modified peptide SP-B₁₋₂₅ (Cys8,11→Ala) was synthesized by standard SPPS Fmoc chemistry on a 0.25 mmol scale using preloaded Wang resin and an ABI 433A automated peptide synthesizer (Table 1). (Merrifield, R. B. (1963) J. Am. Chem. Soc. 85, 2149-2154.) Peptoids were synthesized by the submonomer method using Rink amide resin on a 0.25 mmol scale, and an ABI 433A, with Boc protection of N-(4-aminobutyl)glycine (NLys) (FIG. 1, Table 1). (See, e.g., Zuckermann, R. N., Kerr, J. M., Kent, S. B. H., and Moos, W. H. (1992) J. Am. Chem. Soc. 114, 10646-10647; and U.S. Pat. No. 6,887,845—each of which is incorporated herein by reference in its entirety.)

More specifically, after the removal of the first Fmoc protecting group from the resin with 20% piperidine in N,N-dimethylformamide (DMF) and rinsing of the resin with DMF, the monomer addition cycle was performed by first acetylating the resin with the addition of 1.2 M bromoacetic acid in DMF, followed by N,N-diisopropyl carbodiimide (DIC). The acetylation step was carried out for 45 minutes and then the resin was washed with DMF. The resin-bound halogen was then displaced by 1.0 M primary amine submonomer in N-methylpyrrolidinone (NMP), which was added to the resin and allowed to react for 90 minutes. (The N-substituent of a particular primary amine corresponds to the N-substituent of a glycine residue within a resulting peptoid sequence. Accordingly, as would be understood in the art, N-substituent identity is limited only by the synthetic or commercial availability of a corresponding primary amine and use thereof in peptoid preparation.) The two-step cycle was repeated until the desired length and sequence of the peptoid was obtained, except for the addition of the lysine-like submonomer (NLys), the alkyl submonomers (e.g., Noct), and the proline residue. The displacement step for the Boc- protected NLys submonomer and the Nocd submonomers was extended to 120 minutes while for the addition of the proline residue, a PyBrop activating system was employed. Additionally, due to poor solubility in NMP, the Noct submonomer was dissolved at 0.8 M in dichloromethane:methanol (1:1). After the proline addition, (or, e.g., addition of any other α-amino acid), the Fmoc group was removed with piperidine as before and the peptoid cycle was continued. Peptoid oligomers were cleaved from the resin and deprotected with 90% TFA along with necessary scavengers for 5 minutes.

All molecules were purified according to standard reverse-phase high performance liquid chromatography (RP-HPLC) purification techniques. Final purities were confirmed to be >97% by analytical RP-HPLC and molecular weights were obtained by either electrospray ionization mass spectrometry (ESI/MS) or matrix-assisted laser desorption/ionization time of flight mass spectrometry (MALDI-TOF/MS). Both monomer (˜8794 Da) and dimer (˜17588 Da) peak intensities were observed in the MALDI-TOF/MS spectra for porcine-derived SP-B (data not shown).

### Example 2

### Spectroscopy

Circular dichroism (CD) measurements were acquired on a Jasco J-715 spectropolarimeter (Easton, Md.) in a cylindrical quartz cuvette (Hellma model 121-QS, Forest Hills, N.Y.) with a scan rate of 100 nm mm⁻¹, 0.02 cm path length, 0.2 nm data pitch, 1 nm bandwith, 2 s response, and 100 mdeg sensitivity, from wavelengths (λ) 280-192 nm. Samples were dissolved in methanol from lyophilized powder to a known concentration of ˜15, 30, or 60 μM and run at room temperature. Each presented CD spectrum represents the average of 40 accumulations. Ultraviolet/Visible (UV/Vis) measurements were recorded in double beam mode on a Cary 500 UV-VIS-NIR spectrophotometer (Varian, Palo Alto, Calif.) using quartz cuvettes (Varian), from λ990-190 nm with a scanning rate of 20 nm min⁻¹ and data interval collection of 1 nm. Samples were dissolved in methanol from lyophilized powder to a known concentration of 5, 50, or 100 μM and run at room temperature. Each UV/Vis is sample was run twice.

### Example 3

### Surfactant Sample Preparation

The lipids DPPC, POPG, and PA were individually dissolved in a chloroform/methanol solution (3/1 [v/v]) to a known concentration (˜2 or 4 mg mL⁻¹). Single-lipid solutions were then combined by volume at the ratio of DPPC:POPG:PA, 68:22:9 [wt:wt:wt] and to a known total lipid concentration (˜2 mg mL⁻¹). This well-characterized lipid formulation, the Tanaka Lipids (TL), is considered an adequate mimic of the non-protein (lipid) fraction of LS. The peptides and peptoids were individually dissolved in methanol from a lyophilized powder to a known concentration (1-2 mg mL⁻¹). For the PBS and LWSB/FM studies, the peptides and peptoids were added to the TL lipid solution at 2.16 mol % (˜10-12 absolute wt %, see Table 1), and the final solution was diluted to ˜1 mg lipid mL⁻¹. For comparative purposes, the inclusion of peptide/peptoid at 2.16 mol % corresponds to 10 wt % SP-B₁₋₂₅ relative to the total lipid content (9.1 absolute wt %). Porcine SP-B was dissolved to a known concentration in chloroform/methanol and added to the TL solution at 0.72 mol %, corresponding to roughly 10 wt % of the monomer unit relative to the total lipid content (9.1 absolute wt %). For PBS and LWSB experiments, the standard deviation of the mean (a) was reported.

### Example 4

### Pulsating Bubble Surfactometry

A commercial pulsating bubble surfactometry (PBS) instrument (General Transco, Largo, FL), modified with a direct, real-time imaging system, which has been previously described and validated in detail, was utilized to obtain both static-mode and dynamic-mode data. Samples were dried to a pellet from chloroform/methanol 3/1 [v/v] using a DNA 120 speedvac (Thermo Electron, Holbrook, N.Y.). The pellet was resuspended in buffer (150 mM NaCl, 10 mM HEPES, 5 mM CaCl₂, pH 6.9) to 1.0 mg lipid mL⁻¹, with a known final volume of 70 μL. Samples were mixed with a pipette 20 times, sonicated with a Fisher Model 60 probe sonicator for two 15-second spurts, and then mixed again 20 times to form a dispersed suspension. Samples were then loaded into a small plastic sample chamber (General Transco) using a modified leak-free methodology. (Seurynck, S. L., Brown, N.J., Wu, C. W., Germino, K. W., Kohlmeir, E. K., Ingenito, E. P., Glucksberg, M. R., Barron, A. E., and Johnson, M. (2005) J. Appl. Physiol. 99, 624-633. Putz, G., Goerke, J., Taeusch, H. W., and Clements, J. A. (1994) J. Appl. Physiol. 76, 1425-1431.) The sample chamber was placed in the instrument water bath at room temperature or 37° C. A bubble with a radius of 0.4 mm was formed, and surface area (SA) was monitored throughout the experiment (bubble size gradually increased in both data collection modes, but had a negligible effect on γ).

Static-mode adsorption data were collected for 20 minutes, where the suspension was allowed to adsorb to the bubble surface over time. Adsorption data were smooth fit to a curve in the Kaleidagraph program by applying a Stineman function to the data, where the output of this function then had a geometric weight applied to the current point and ±10% of the data range to arrive at the smoothed curve. Dynamic-mode data were then subsequently obtained for each sample at the adult respiratory cycle frequency of 20 cycles per minute (cpm) for 10 minutes, with a 50% reduction in surface area per pulsation cycle. PBS experiments were repeated six times for unlabeled films at 37° C., and three times for fluorescently labeled films at 37° C. and all films at room temperature. Representative PBS loops are presented at five minutes of cycling, and indicate clockwise bubble expansion from left to right. Percent compression is defined here as 100*[(SAₘₐₓ−SA₂₀)/(SAₘₐₓ)], where SAₘₐₓ was the maximum SA value at expansion, and SA₂₀ was the SA at which γ first reaches 20 mN m⁻¹ upon compression.

### Example 5

### Langmuir-Wilhelmy Surface Balance and Epifluorescence Microscopy Studies

Surface pressure (π)-molecular area (A) isotherms were obtained using a custom-built Langmuir-Wilhelmy surface balance (LWSB), where the instrument and method have been previously described in detail. (Seurynck, S. L., Patch, J. A., and Barron, A. E. (2005) Chem. Biol. 12, 77-88. Seurynck-Servoss, S. L., Dohm, M. T., and Barron, A. E. (2006) Biochemistry 45, 11809-11818. Seurynck-Servoss, S. L., Brown, N. J., Dohm, M. T., Wu, C. W., and Barron, A. E. (2007) Coll. Surf B. Biointerfaces 57, 37-55. Brown, N. J., Wu, C. W., Seurynck-Servoss, S. L., and Barron, A. E. (2008) Biochemistry 47, 1808-1818.) The trough was filled with 300 mL of aqueous buffer (150 mM NaCl, 10 mM HEPES, 5 mM CaCl₂, pH 6.9) and heated to 25 or 37° C. A Wilhelmy plate (Reigler & Kirstein GMBH, Berlin, Germany) was used to monitor surface pressure and calibrated in buffer before each run. Each sample was spread at the all interface from a 3/1 chloroform/methanol solution [v/v] using a glass syringe and allowed to equilibrate for 5-10 minutes. The barriers were then compressed, expanded, and compressed again at a rate of 30 mm mm⁻¹. Isotherm measurements were repeated at least six times for each sample.

To record epifluorescence microscopy (FM) images, a Nikon MM40 compact microscope stand with a 100W mercury lamp (Tokyo, Japan) was used in conjunction with the LWSB. Epifluorescence was detected by a Dage-MTI three-chip color camera (Dage-MTI, Michigan City, Ind.) in conjunction with a generation II intensifier (Fryer, Huntley, Ill.). Samples were spiked with 0.50 mol % TR-DHPE, a fluorescently headgroup-labeled lipid, for detection. Isotherm features remained unchanged after TR-DHPE addition, and presumably, film morphology was also unchanged by the presence of TR-DHPE at these concentrations. FM images were acquired directly from the compressed film on the all interface. Experiments were conducted exactly as the LWSB studies of unspiked films, with the exception that barrier speed was reduced to 5 mm mm⁻¹, and experiments were repeated three times. Average liquid-condensed (LC) domain sizes were calculated using ImageJ software (National Institutes of Health, Bethesda, Md.).

Examples 16-15, in particular, can be considered in conjunction with in vivo treatments and related methodologies of this invention.

### Example 6

### Peptoid Synthesis and Purification

Peptoids were synthesized using an ABI 433A peptide synthesizer (Foster City, Calif.) on Rink amide resin (NovaBiochem, San Diego, Calif.) as described, above, using peptoid synthesis reagents (Sigma-Aldrich (Milwaukee, Wis.), solvents (Fisher Scientific (Pittsburgh, Pa.), and side-chain primary amines benzylamine, octadecylamine, isopropylamine, isobutylamine, S-alpha methylbenzylamine, N-tert-butoxycarbonyl-1,4 diaminobutane, and L-proline. Resin-bound peptoids were cleaved in an trifluoroacetic acid-scavenger mixture and purified by standard reversed-phase HPLC (RP-HPLC) (C4 or C18 column, linear acetonitrile/water gradient). Analytical RP-HPLC purity was >97%; and molar masses were confirmed using electrospray ionization mass spectrometry (ESI/MS).

### Example 7

### Preparation of Surfactant Mixtures

Dipalmitoyl phosphatidylcholine (DPPC) and palmitoyoleol phosphatidylglycerol (POPG) (Avanti Polar Lipids, Alabaster, Ala.), palmitic acid (PA) (Sigma-Aldrich), and solvents (Fisher Scientific) comprised the Tanaka lipid formulation (22.5 mg Tanaka lipids per animal), which was prepared in a glass vial by combining individual lipid stock solutions to yield a 68:22:9 (w:w:w) mixture of DPPC:POPG:PA in chloroform/methanol (3:1 v:v) solution. Peptoid was added to the lipids from methanol stock solutions at ˜2 mol % peptoid (˜10 wt % relative to total lipid content), and in two-peptoid formulations, 1 mol % per peptoid. Surfactant mixtures were dried under nitrogen, lyophilized, and stored at −20° C. Adding sterile saline (25 mg/mL) and resuspending provided a homogenous, flowable lipid-peptoid surfactant composition.

### Example 8

Animal Experiments. Procedures were approved by AUS at the University of Western Ontario, London Ontario, Canada, according to the CCAC. Sprague-Dawley rats (360-410 g) (Charles River, St. Constant, PQ, Canada) were weighed, anesthetized by intraperitoneal (i.p.) injection (75 mg/kg Ketamine Hydrochloride, 5 mg/kg Xylazine, sterile 0.15 M saline), and given 0.05-0.1 mg/kg of buprenorphine subcutaneously. Right carotid artery access via isolation and cannulation permitted blood gas sampling, measuring vital signs, and instilling fluids (0.15 M saline, 0.5 mL/kg/hour), while similarly obtained jugular venous line access allowed administering of ˜1.0 mg/kg/min Propofol and fluids. Following tracheostomy and endotracheal tube placement, pancuronium bromide (1 mg/kg i.v.) was administered to inhibit spontaneous movement. Ventilator settings were: tidal volume, 8 mL/kg; positive end expiratory pressure (PEEP), 5 cm H₂O; respiratory rate, 55-60 breaths/minute; and FiO₂, 1.0 (volume-cycle mechanical rodent ventilator, Harvard Instruments, St. Laurent, PQ, Canada; airway pressure monitor, Caradyne Ltd, Indianapolis, Ind.). Initial inclusion criterion was baseline PaO₂>400 mmHg.

Whole lung lavage was performed. After ventilator removal, 0.15 M NaCl (10 mL, 37° C.) was instilled/withdrawn from the lungs, followed by mechanical ventilation. Lungs were lavaged four times, 5 minutes apart preceding a blood gas measurement. Study inclusion required PaO₂<120 mmHg. Non-inclusive animals were re-lavaged until the inclusion criterion was satisfied.

Animals were randomized into five treatment groups: 1) bovine lipid extract surfactant (BLES, BLES Biochemicals, London Ontario, CA) (positive control, n=7), 2) surfactant composition pB (n=6), 3) surfactant composition pC (n=7), 4) surfactant composition pB/pC (n=7), 5) Tanaka lipids (negative control, n=5). After ventilator removal, upright animals were instilled with a 50 mg/kg surfactant bolus endotracheally via syringe, and then a 3 mL air bolus that ensured distribution to distal regions. Ventilation and monitoring occurred for 2 hours, with blood gas sampling at semi-regular timepoints. Recovery vital signs were monitored for adequate perfusion, as was anesthestic state. Measured physiological responses included PaO₂, blood pH, shunt fraction, A-a gradient, and peak inspiratory pressure (PIP). Post-experiment, animals were euthaniZed via sodium pentobarbital, exsanguinated, the chest wall opened, lung-lavaged five times, and total lavage volume was recorded.

### Example 9

Surfactant Analysis. After broncheoalveolar lavage fluid (BAL) centrifugation (150 g, 10 minutes), 5 mL of supernatant “Total Surfactant (TS)” was aliquoted for further analysis, and the remainder centrifuged (40,000 g, 15 minutes) to separate supernatant Small Aggregates (SA) from pellet. Resuspended pellets (in 2 mL saline) produced Large Aggregates (LA). Aliquots were extracted (Bligh/Dyer method), phospholipids quantified (Duck-Chong phosphorous assay, Duck-Chong CG. Rapid sensitive method for determining phospholipid phosphorus involving digestion with magnesium nitrate. Lipids 1979; 14:492-497), and BAL total protein content determined (micro-BCA protein assay, Pierce Biotechnology, Rockford, Ill.).

### Example 10

Statistical Analysis. Presented data are means±SEM and were analyzed via one-way ANOVA using the Tukey-Kramer method (p<0.05).

### Example 11

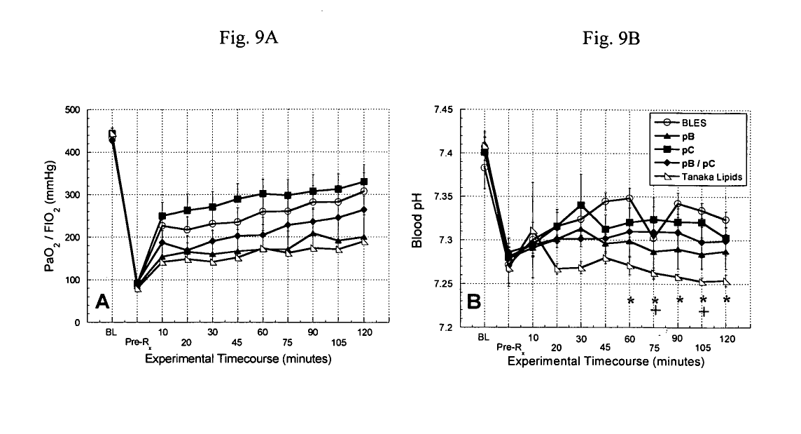

Physiological Responses. In general, the surgical procedure was well-tolerated by the animals. Three animals died during the procedure, and 13 animals did not meet the study inclusion criteria. For the 32 animals included in the study, the average baseline blood oxygen level (PaO₂), normalized to the fraction of inspired oxygen (FIO₂) (1.0 throughout all experiments), of 435.7±4.9 mm Hg was reduced to 88.3±2.5 mm Hg post-lavage, reflective of surfactant deficiency (FIG. 9A). Similarly, the average baseline peak inspiratory pressure (PIP) of 12.0±0.3 cm H₂O was increased to 20.8±0.4 cm H₂O post-lavage. (The average blood pressure and heart rate for each treatment group over the time course of the experiments are shown in FIGS. 10A-B.)

### Example 12

The average PaO₂/FIO₂ as a function of time is shown for each treatment group in FIG. 9A. The immediate response to surfactant treatment is reflected in changes from the pre-treatment (Pre-Rx) condition to the 10 minute data time point. The ability of surfactant treatments to sustain a response was gleaned by comparing the 10 minute time point data to conditions at the end of the two hour observation period. Animals treated with the positive control BLES (p<0.001), pC (p<0.0005), and pB/pC (p<0.007) showed a statistically significant, immediate improvement in oxygenation upon treatment. The immediate improvements demonstrated by the negative control Tanaka lipids group (p<0.23) and pB group (p<0.07) were not significant. Treatment with BLES (p<0.16), pC (p<0.15), and pB/pC (p<0.11) also demonstrated better sustained oxygenation throughout the two hour observation period compared with the pB (p<0.47) and Tanaka lipid (p<0.60) treatment groups. Animals in the pC treatment group were correlated with the highest arterial blood oxygenation levels throughout the study.

### Example 13

The blood pH as a function of time is shown for each treatment group in FIG. 9B. Comparing baseline to pre-treatment conditions, pulmonary lavage caused a significant (p<0.005) and uniform lowering of the blood pH in all treatment groups. On average, the highest blood pH outcome was achieved by the BLES treatment group, which was found to be statistically different (p<0.05) from the Tanaka lipids treatment group at t>45 minutes. Among peptoid-enhanced surfactants, treatment with pC exhibited the most complete return to baseline conditions; the blood pH of this treatment group was statistically different (p<0.05) from the Tanaka lipids treatment group at 75 and 105 minutes.

### Example 14

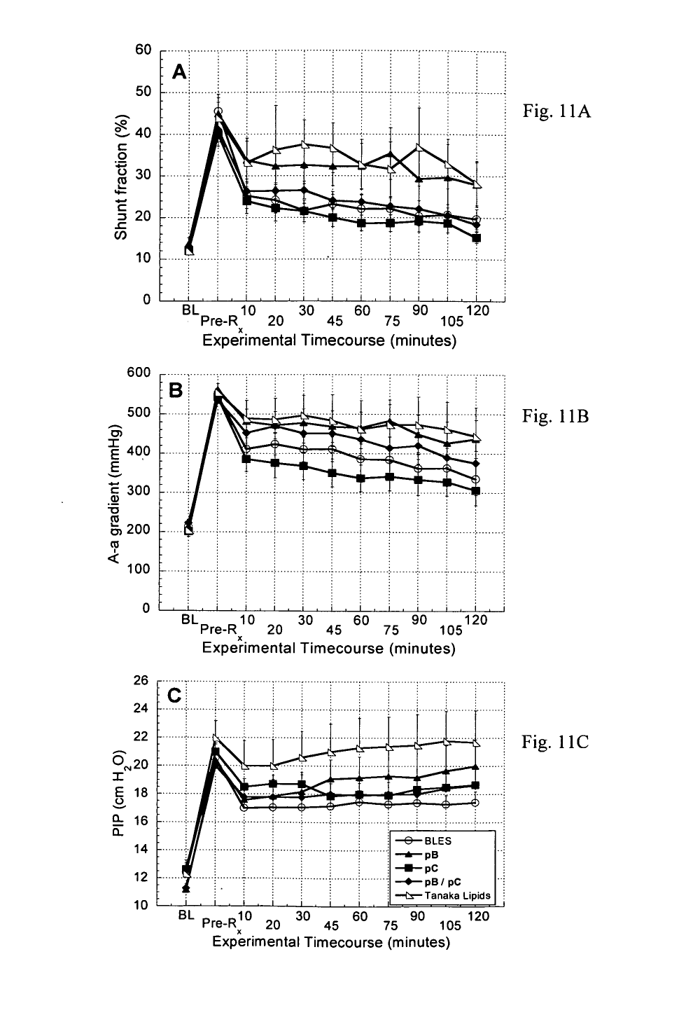

FIG. 11 displays three additional indicators of pulmonary function, including shunt fraction, A-a gradient, and PIP. Ten minutes after treatment, the shunt fraction decreased significantly for the BLES (p<0.0005), pC (0.001), and pB/pC (p<0.01). The further decrease in shunt fraction observed from the 10 minute time point until the end of the two hour observation period was statistically significant for the pC (p<0.05) and pB/pC (p<0.05) treatment groups. As shown in FIG. 11A, the pC treatment group was shown to be statistically different (p<0.05) from the pB and Tanaka lipids treatment groups at selected timepoints. The A-a gradient data shown in FIG. 11B exhibits a statistically significant, immediate response for all treatment groups (p<0.05) except Tanaka lipids (p<0.20). The pC treatment group resulted in the most significant immediate decrease (p<0.0003), but the pB/pC treatment group resulted in the best sustained response from the 10 minute time point throughout the observation period (p<0.1). The PIP data shown in FIG. 11A demonstrated statistically significant immediate improvement at 10 minutes for BLES (p<0.05) and pC (p<0.01) (pB and pB/pC exhibited p<0.1). The Tanaka lipid treatment group immediate improvement was not significant (p<0.2). No treatment groups demonstrated sustained improvement of PIP throughout the 2 hour observation period.

### Example 15

Surfactant Pool Evaluation. Since the efficacy of surfactant may be influenced by its metabolism within the airspace, phospholipid pools and total protein content in the broncheoalveolar lavage (BAL) fluid from each animal were evaluated at the end of the ventilation period. The average amount of total surfactant (TS), large aggregates (LA), and small aggregates (SA) obtained from the BAL of each treatment group is shown in FIG. 12A. While there was no statistically significant difference between the large aggregate contents of the treatment groups, the amount of small aggregates was higher in the Tanaka lipid treatment group than in any other (p<0.05). The average amount of total surfactant was highest for the Tanaka lipids treatment group, and statistically higher (p<0.05) than the pC treatment group. The average total protein content of the BAL for each treatment group is shown in FIG. 12B. The data show that there was no statistically significant difference in the total protein content among the various treatment groups.

While the principles of this invention have been described in connection with specific embodiments, it should be understood clearly that these descriptions are added only by way of example and are not intended to limit, in any way, the scope of this invention. For instance, any one or more of the peptoid compounds of this invention can be dimerized through incorporation of cysteine residues at or about either terminus or therebetween to provide disulfide bond capability and subsequent dimer linkage. Various other chemistries, as would be understood by those skilled in the art, could also be used with comparable effect. Likewise, one or more amino acids or N-substituted residues can be introduced to provide sites for later, in situ enzymatic cleavage. Other structural and/or functional variations of the present peptoid compounds will be understood by those skilled in the art and made aware of this invention.

Likewise, any one or more of the peptoid compounds of this invention can be incorporated into a lung surfactant composition, such compositions as can optionally comprise one or more synthetic or naturally derived surfactants proteins, lipids and/or fatty acids. While several such compositions are formulated as described herein, it will be understood by those skilled in the art that such formulations and effective dosages or concentrations are limited only by sufficient administration and corresponding treatment of a pulmonary disorder. An effective dosage will be understood by those skilled in the art and can be determined in accordance with the guidelines/parameters and indications demonstrated herein. Generally, administration can be tracheally, local or as otherwise designed to target an alveolar network and/or a corresponding air/liquid interface. Accordingly, compositions of this invention can be formulated as part of a solution, an emulsion, a suspension, a bolus and the like for delivery and/or administration by deposition, injection, aerosol spray or any other technique known in the art. Other formulations and/or delivery techniques, for the present compositions, will be understood by those skilled in the art made aware of this invention.

We claim:

1. A poly-N-substituted glycine compound of a formula
A-B—X—Y—Z
wherein A is selected from H and N-alkyl substituted glycine residues, where said alkyl substituent is selected from about C₄- about C₂₄ linear, branched and cyclic alkyl moieties; B is selected from N-alkyl substituted glycine residues, where said alkyl substituent is selected from C₄- about C₂₄ linear, branded and cyclic alkyl moieties; X is a component comprising about 3- about 9 N-substituted glycine residues, said X component comprising a plurality of Nₛₚₑ residues; Y is a component comprising about 3- about 23 N-substituted glycine residues, said Y component comprising residues selected from N_{Lys} and Nₛₚₑ residues, providing at least one of said residues is N_{Lys}; and Z is a C-terminus selected from NH₂, one and two N-substituted glycine residues selected from N_{Lys} and Nₛₚₑ.

2. The compound of claim 1 wherein each of A and B is independently selected from N-alkyl substituted glycine residues.

3. The compound of claim 2 wherein each said alkyl substituent is independently selected from linear C₈-C₂₀ alkyl moieties.

4. The compound of claim 1 wherein each said X residue is an Nₛₚₑ residue.

5. The compound of claim 4 where said X component is 6-7 residues.

6. The compound of claim 1 wherein said Y component is 10-14 residues comprising a combination of N_{Lys} and Nₛₚₑ residues.

7. The compound of claim 6 wherein 4-6 of said residues are N_{Lys}.

8. The compound of claim 1 wherein each said X and Y components independently comprise at least one other residue selected from proline and N-substituted glycine residues, said N-substituents independently selected from α-amino acid side chain moieties and carbon homologs thereof.

9. The compound of claim 8 wherein X and Y independently comprise residues selected from Nₚₘ, N_{ssb}, N_{sdp}, N_{Leu}, N_{Ile}, Nₚₕₑ, N_{Trp}, and N_{Tyr}, and combinations thereof.

10. The compound of claim 8 wherein Y comprises an N_{Arg} residue.

11. The compound of claim 1 wherein said X component is 6-7 residues, said Y component is 10-14 residues and Z is NH₂.

12. The compound of claim 1 in a lung surfactant composition.

13. A poly-N-substituted glycine compound of a formula
A-B—X—Y—Z₂—Y₂—Z₄—Y—Z₂—C
wherein A is selected from H and N-alkyl substituted glycine residues, where said alkyl substituent is selected from about C₄- about C₂₄ linear, branched and cyclic alkyl moieties; B is selected from N-alkyl substituted glycine residues, where said alkyl substituent is selected from C₄- about C₂₄ linear, branded and cyclic alkyl moieties; X is a component comprising 6-7 N-substituted glycine residues, said X component comprising a plurality of Nₛₚₑ residues; each said Y component independently comprising a residue selected from N_{Lys} and N_{Arg} residues and combinations thereof; each said Z component independently comprises residues selected from Nₛₚₑ, Nₚₘ, N_{ssb}, N_{sdp}, N_{Leu}, N_{Ile}, Nₚₕₑ, N_{Trp}, N_{His} and N_{Tyr}, and combinations thereof; and C is a terminal moiety comprising NH₂.

14. The compound of claim 13 wherein said X and Y components independently comprise residues selected from Nₚₘ, N_{ssb}, N_{sdp}, N_{Leu}, N_{Ile}, Nₚₕₑ, N_{Trp}, N_{His} and N_{Tyr}, and combinations thereof.

15. The compound of claim 13 wherein said X component is a sequence of 6-7 Nₛₚₑ residues.

16. The compound of claim 15 selected from Nₒₑₜ-Nₛₚₑ₇-N_{Lys}-Nₛₚₑ₂-N_{Lys2}-Nₛₚₑ₄-N_{Lys}-Nₛₚₑ₂ and N_{oct2}-Nₛₚₑ₆-N_{Lys}-Nₛₚₑ₂-N_{Lys2}-Nₛₚₑ₄-N_{Lys}-Nₛₚₑ₂.

17. A lung surfactant composition comprising a biomimetic SP-B compound of claim 1, and a lipid component selected from naturally-derived phospholipids, non-natural phospholipids, commercial surface-active agents and combinations thereof.

18. The composition of claim 17 comprising a compound of claim 13.

19. The composition of claim 18 wherein X and Y independently comprise residues selected from Nₚₘ, N_{ssb}, N_{sdp}, N_{Leu}, N_{Ile}, Nₚₕₑ, M_{Trp}, N_{His} and N_{Tyr}, and combinations thereof.

20. The composition of claim 19 wherein said biomimetic SP-B compound is selected from N_{oct}-Nₛₚₑ₇-N_{Lys}-Nₛₚₑ₂-N_{Lys2}-Nₛₚₑ₄-Nₛₚₑ₄-N_{Lys}-Nₛₚₑ₂ and N_{oca}-Nₛₚₑ₆-N_{Lys}-Nₛₚₑ₂-N_{Lys2}-Nₛₚₑ₄-Nₛₚₑ₄-N_{Lys}-N_{spe2.}

21. The composition of claim 17 wherein said lipid component comprises DPPC, POPG and PA.

22. The composition of claim 17 comprising a component selected from naturally-derived SP-C components, biomimetic SP-C components and combinations thereof.

23. The composition of claim 22 wherein said biomimetic SP-B compound is selected from N_{oct}-Nₛₚₑ₇-N_{Lys}-Nₛₚₑ₂-N_{Lys2}-Nₛₚₑ₄-Nₛₚₑ₄-N_{Lys}-Nₛₚₑ₂ and N_{oct2}-Nₛₚₑ₆-N_{Lys}-Nₛₚₑ₂-N_{Lys2}-Nₛₚₑ₄-Nₛₚₑ₄-N_{Lys}-Nₛₚₑ₂.

24. The composition of claim 22 wherein said biomimetic SP-B compound and a said SP-C component comprise up to about 20 wt. % of said composition.

25. A method of treating a mammalian pulmonary disorder, said method comprising:
providing a mammalian subject exhibiting a physiological condition comprising a lung surfactant deficiency; and
administering a composition of claim 17 to said subject, said composition in a therapeutically effective amount.

26. The method of claim 25 wherein said composition comprises a component selected from naturally-derived SP-C components, biomimetic SP-C components and combinations thereof.

27. The method of claim 26 wherein said composition comprises a compound of claim 13.

28. The method of claim 27 wherein said compound is selected from N_{oct}-Nₛₚₑ₇-N_{Lys}-Nₛₚₑ₂-N_{Lys2}-Nₛₚₑ₄-N_{Lys}-Nₛₚₑ₂Nₛₚₑ₄-N_{Lys}-Nₛₚₑ₂ and N_{oct2}-Nₛₚₑ₆-N_{Lys}-Nₛₚₑ₂-N_{Lys2}-Nₛₚₑ₄-Nₛₚₑ₄

29. The method of claim 25 wherein said composition is formulated as a liquid bolus or an aerosol spray.

30. The method of claim 25 wherein said composition is administered via the trachea of said subject.
